(19) United States
(12) Patent Application Publication
(10) Pub. No.: US 2025/0082664 A1
(43) Pub. Date: Mar. 13, 2025

(54) NUCLEOSIDE FORMULATION

(71) Applicant: PHION THERAPEUTICS LTD, Belfast (GB)

(72) Inventors: Helen MCCARTHY, Belfast (GB); Lindsey Ann BENNIE, Belfast (GB); Philip CHAMBERS, Belfast (GB)

(73) Assignee: PHION THERAPEUTICS LTD., Belfast (GB)

(21) Appl. No.: 18/720,494

(22) PCT Filed: Dec. 15, 2022
(86) PCT No.: PCT/EP2022/086083

§ 371 Date: Jun. 14, 2024

(30) Foreign Application Priority Data
Dec. 16, 2021 (GB) 2118225.8

(51) Int. Cl. A61K 31/7068; A61K 9/00; A61K 47/36; A61K 47/64; A61K 47/69; A61P 35/00

(52) U.S. Cl. CPC A61K 31/7068; A61K 9/0019; A61K 47/36; A61K 47/6455; A61K 47/6929; A61P 35/00

(57) ABSTRACT

The present specification relates to a nucleoside formulation which comprises a nucleoside or a phosphate thereof; and an amphipathic cell penetrating RALA peptide. In one embodiment, the nucleoside formulation comprises gemcitabine or a phosphate thereof and the peptide WEARLARALARALARHLARALARALRACEA.

### FIELD

The present specification relates to a nucleoside formulation which comprises a nucleoside or a phosphate thereof; and an amphipathic cell penetrating peptide from the RALA family of peptides. The present further specification relates to methods of preparing nucleoside formulations and their use in therapy.

### BACKGROUND

The nucleoside analogues are an important class of antiviral agents now commonly used in the treatment of viral infections and cancer. Once in vivo, nucleoside analogues are phosphorylated, and incorporated into growing DNA strands acting as chain terminators.

There are three main problems with nucleoside analogues as illustrated by those incurred with gemcitabine: 1) damaging side effects, such as bone marrow suppression, liver and kidney problems, and nausea, limit the dose that can be given to patients because the drug is distributed widely in the body and enters other, non-tumour, cells; 2) an extremely short drug stability and half-life, measured in minutes, requires high dose infusions further adding to the toxicity risks; and 3) drug resistant mutations quickly appear leading to a low overall clinical response rate.

The nucleoside analogue gemcitabine is a chemotherapy drug used to treat a number of different types of cancer, first approved for use in 1995. It is prescribed as a first line therapy for pancreatic cancer, and for the treatment of other cancers where patients have failed previous therapies. The 5 year survival rate of pancreatic cancer is still only about 9% (Office for National Statistics, Cancer survival by stage at diagnosis for England, 2019) and it is the cause of nearly 450,000 deaths a year worldwide, accounting for 4.5% of all cancer deaths (GLOBOCAN 2018 estimates). Once in the body, gemcitabine is transported into cells via molecular transporters. It is then phosphorylated three times to produce the pharmacologically active form, gemcitabine triphosphate (dFdCTP). This is then incorporated into DNA as the cancerous cell replicates creating an irreparable error, leading to inhibition of further DNA synthesis and ultimately cell death.

Unfortunately, gemcitabine has a low overall clinical response rate of 6% (Ioka T, et al. Jpn J Clin Oncol. 2013 February; 43(2):139-45). Furthermore, multiple drug resistance mechanisms have been identified relating to mutation of the active transporters needed for cellular uptake; upregulation of metabolic enzymes that inactivate gemcitabine; and downregulation of cellular enzymes that convert gemcitabine to the active triphosphate form (Kim H A, et al. Breast. 2008 February; 17(1):19-26).

There is clearly a need for formulations of nucleoside analogues that will improve cell entry, by-pass resistance mechanisms, increase accumulation, and improve half-life and drug stability. Furthermore, a drug delivery system that could deliver the active, e.g. tri-phosphate, forms into the cell of drugs such as gemcitabine would potentially automatically bypass some of the mechanisms that lead to drug resistance (Galmarini C M, et al. Int J Pharm. 2010 Aug. 16; 395(1-2):281-9).

The RALA family of peptides are amphipathic peptides composed of repeating RALA units that are capable of overcoming biological barriers to gene delivery, both in vitro and in vivo. The term “RALA” has been used inconsistently in the literature, but typically refers to an amphipathic peptide or group of peptides composed of repeating RALA units generally of less than approximately 50 amino acid residues. Cohen-Avrahami M et al. (J. Phys. Chem. B 2011, 115:10 189-1 097 and Colloids Surf B Biointerfaces. 2010 Jun. 1; 77(2):131-8) disclose an amphipathic 16-mer peptide referred to as “RALA”. Nouri F S et al. (Biomacromolecules 2013, 14, 2033-40) uses the term “RALA” to describe a 30-mer RALA peptide, as does McCarthy H O et al. (J Control Release. 2014 Sep. 10; 189:141-9) but for a different 30-mer peptide. WO 2014/087023 and WO 2015/189205 defined the term “RALA” as a generic term for a group of peptides falling within the scope of the invention as described therein.

The RALA family of peptides have been used to deliver genetic material such as plasmid DNA (McCarthy H O, et al., J Control Release. 2014 Sep. 10; 189:141-9) and (Ali A A, et al., Nanomedicine. 2017 April; 13(3):921-932), mRNA (Udhayakumar et al., Adv Healthc Mater. 2017 July; 6(13)), siRNA (Mulholland E J, et al., J Control Release. 2019 Dec. 28; 316:53-65), and small molecules such as bisphosphonates (Jena L N, et al., J. Nanobiotechnology. 2021 May 4; 19(1):127), and calcium phosphates (Sathy B N et al., J. Mater. Chem. B. 2017 Mar. 7; 5(9):1753-1764).

The specific RALA peptide WEARLARALARALARHLARALARALRACEA (SEQ ID_No 1) is a 30 amino acid, non-toxic, peptide with a +6 electric charge at a physiological pH that converts to a +8 helical cell penetrating conformation at acidic conditions found inside the endosome of a cell. When complexed with certain payloads, e.g. DNA and mRNA, in water it has been shown to be capable of spontaneous self-assembly into nanoparticles (McCarthy H O, et al., J Control Release. 2014 Sep. 10; 189:141-9 and Udhayakumar et al., Adv Healthc Mater. 2017 July; 6(13)). This pH dependent change allows for escape of the peptide and the cargo within a cell, resulting in highly efficiency cellular entry and cargo delivery, without any associated toxicity at the physiological pH of 7.4 outside the cell. The nanoparticles have been shown to be extremely stable at a range of temperatures and over time, and RALA peptides do not themselves provoke an immunological response.

The present application describes an improved formulation of nucleosides and/or their phosphates, formulating them with an amphipathic cell penetrating peptide from the RALA family of peptides. The formulation is capable of spontaneously forming nanoparticles when mixed in water. The formulations are capable of improved target selective delivery with the potential to reduce off-target toxicity while improving pharmacokinetics, enhancing pharmacodynamics, and bypassing drug resistance mechanisms, facilitating a more potent therapeutic effect with a lower dose. Such improved properties may open up the possibility of using the nucleoside analogue family of compounds and their phosphates for new indications. Where the nucleoside or a phosphate thereof is the pharmacologically active form of gemcitabine, gemcitabine triphosphate, the nanoparticles possess superior tumour selective delivery when compared to existing gemcitabine formulations, with a longer circulatory half-life, and result in a 2-fold higher drug accumulation in solid tumours following intravenous administration when compared to other sites in vivo.

### SUMMARY

This specification describes, in part, a nucleoside formulation comprising a nucleoside or a phosphate thereof; and an amphipathic cell penetrating peptide comprising or consisting of the amino acid sequence WEARLARALARALARHLARALARALRACEA (SEQ ID_No 1) or a sequence with at least 80% sequence identity or homology.

This specification also describes, in part, a nucleoside formulation comprising gemcitabine or a phosphate thereof; and an amphipathic cell penetrating peptide comprising or consisting of the amino acid sequence WEARLARALARALARHLARALARALRACEA (SEQ ID_No 1) or a sequence with at least 80% sequence identity or homology.

This specification also describes, in part, a nucleoside formulation as described herein, wherein the formulation is a nanoparticle formulation, and methods of preparing them.

This specification also describes, in part, nucleoside formulations as described herein for use in therapy, particularly for use in the treatment of viral infections and in the treatment of cancer.

### DETAILED DESCRIPTION OF THE INVENTION

Many embodiments of the invention are detailed throughout the specification and will be apparent to a reader skilled in the art. The invention is not to be interpreted as being limited to any of the recited embodiments.

“A” means “at least one”. In any embodiment where “a” is used to denote a given material or element, “a” may mean one.

“Comprising” means that a given material or element may contain other materials or elements. In any embodiment where “comprising” is mentioned the given material or element may be formed of at least 10% w/w, at least 20% w/w, at least 30% w/w, or at least 40% w/w of the material or element. In any embodiment where “comprising” is mentioned, “comprising” may also mean “consisting of” (or “consists of”) or “consisting essentially of” (or “consists essentially of”) a given material or element.

“Consisting of” or “consists of” means that a given material or element is formed entirely of the material or element. In any embodiment where “consisting of” or “consists of” is mentioned, the given material or element may be formed of 100% w/w of the material or element.

“Consisting essentially of” or “consists essentially of” means that a given material or element consists almost entirely of that material or element. In any embodiment where “consisting essentially of” or “consists essentially of” is mentioned the given material or element may be formed of at least 50% w/w, at least 60% w/w, at least 70% w/w, at least 80% w/w, at least 90% w/w, at least 95% w/w or at least 99% w/w of the material or element.

In any embodiment where “is” or “may be” is used to define a material or element, “is” or “may be” may mean the material or element “consists of” or “consists essentially of” the material or element.

Claims are embodiments.

### Nucleoside

A nucleoside comprises a nitrogenous base and a five-carbon sugar.

In one embodiment the nucleoside is a monomeric unit, not polymerised into a longer chain.

In one embodiment the nucleoside is not RNA or DNA.

In one embodiment the nucleoside comprises a nitrogenous base selected from adenine, guanine, thymine, uracil and cytosine or a modified version thereof.

A modified version of a nitrogenous base may include, for example the addition, substitution or removal of one or more halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl or cyclopropyl group, or the rearrangement and/or removal and/or addition of further heteroatoms to the nitrogenous base.

In one embodiment the nucleoside comprises a nitrogenous base selected from adenine or a modified version thereof.

In one embodiment the nucleoside comprises a nitrogenous base selected from guanine or a modified version thereof.

In one embodiment the nucleoside comprises a nitrogenous base selected from thymine or a modified version thereof.

In one embodiment the nucleoside comprises a nitrogenous base selected from uracil or a modified version thereof.

In one embodiment the nucleoside comprises a nitrogenous base selected from cytosine or a modified version thereof.

In one embodiment the nucleoside comprises a five-carbon sugar selected from ribose or 2′-deoxyribose or a modified version thereof.

A modified version of a five-carbon sugar may include, for example the addition, substitution or removal of one or more halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl, N-methyl-N-ethylsulphamoyl or cyclopropyl group, or the substitution of one or more carbons in the five-carbon sugar for one or more heteroatoms, for example sulphur.

In one embodiment the nucleoside comprises a five-carbon sugar selected from ribose or a modified version thereof.

In one embodiment the nucleoside comprises a five-carbon sugar selected from 2′-deoxyribose or a modified version thereof.

In one embodiment the nucleoside is a “nucleoside analogue” from the nucleoside analogue family of pharmaceutically active agents.

In one embodiment, a nucleoside or a phosphate thereof refers to a deoxyadenosine, adenosine, deoxycytidine, guanosine, deoxyguanosine, thymidine, deoxythymidine or deoxyuridine analogue or a phosphate thereof.

In one embodiment, a nucleoside or a phosphate thereof refers to didanosine, vidarabine, galidesivir, remdesivir, cytarabine, gemcitabine, emtricitabine, lamivudine, zalcitabine, abacavir, aciclovir, entecavir, stavudine, telbivudine, zidovudine, idoxuridine or trifluridine or a phosphate thereof.

In one embodiment, a nucleoside or a phosphate thereof refers didanosine or a phosphate thereof. Didanosine or a phosphate thereof is useful in the treatment and/or prevention of HIV and AIDS.

In one embodiment, a nucleoside or a phosphate thereof refers to vidarabine or a phosphate thereof. Vidarabine or a phosphate thereof is useful in the treatment and/or prevention of herpes simplex and varicella zoster viruses.

In one embodiment, a nucleoside or a phosphate thereof refers to galidesivir or a phosphate thereof. Galidesivir or a phosphate thereof is useful in the treatment and/or prevention of hepatitis C, Ebola virus, Marburg virus, Zika virus and coronavirus.

In one embodiment, a nucleoside or a phosphate thereof refers to remdesivir or a phosphate thereof. Remdesivir or a phosphate thereof is useful in the treatment and/or prevention hepatitis C, Ebola virus, Marburg virus, and as a post-infection treatment for COVID-19.

In one embodiment, a nucleoside or a phosphate thereof refers to cytarabine or a phosphate thereof. Cytarabine or a phosphate thereof is useful in the treatment and/or prevention of acute myeloid leukemia (AML), acute lymphocytic leukemia (ALL), chronic myelogenous leukemia (CML), and non-Hodgkin's lymphoma.

In one embodiment, a nucleoside or a phosphate thereof refers to gemcitabine or a phosphate thereof. Gemcitabine or a phosphate thereof is useful in the treatment and/or prevention of testicular cancer, breast cancer, ovarian cancer, non-small cell lung cancer, pancreatic cancer, and bladder cancer; particularly metastatic breast cancer; or first-line treatment of metastatic breast cancer after failure of adjuvant chemotherapy; or the first-line treatment of patients with inoperable, locally advanced (Stage IIIA or IIIB) or metastatic (Stage IV) non-small cell lung cancer; or the treatment of locally advanced (nonresectable Stage II or Stage III) or metastatic (Stage IV) adenocarcinoma of the pancreas; or the treatment of locally advanced or metastatic bladder cancer; or the treatment of locally advanced or metastatic epithelial ovarian carcinoma.

In one embodiment, a nucleoside or a phosphate thereof refers to emtricitabine or a phosphate thereof. Emtricitabine or a phosphate thereof is useful in the treatment and/or prevention of HIV infection.

In one embodiment, a nucleoside or a phosphate thereof refers to lamivudine or a phosphate thereof. Lamivudine or a phosphate thereof is useful in the treatment and/or prevention of HIV/AIDS and chronic hepatitis B.

In one embodiment, a nucleoside or a phosphate thereof refers to zalcitabine or a phosphate thereof. Zalcitabine or a phosphate thereof is useful in the treatment and/or prevention of HIV/AIDS.

In one embodiment, a nucleoside or a phosphate thereof refers to abacavir or a phosphate thereof. Abacavir or a phosphate thereof is useful in the treatment and/or prevention of HIV/AIDS.

In one embodiment, a nucleoside or a phosphate thereof refers to aciclovir or a phosphate thereof. Aciclovir or a phosphate thereof is useful in the treatment and/or prevention of herpes simplex virus infections, varicella zoster virus infections, cytomegalovirus infections and severe complications of Epstein-Barr virus infection.

In one embodiment, a nucleoside or a phosphate thereof refers to entecavir or a phosphate thereof. Entecavir or a phosphate thereof is useful in the treatment and/or prevention of hepatitis B virus (HBV) infection.

In one embodiment, a nucleoside or a phosphate thereof refers to stavudine or a phosphate thereof. Stavudine or a phosphate thereof is useful in the treatment and/or prevention of HIV/AIDS.

In one embodiment, a nucleoside or a phosphate thereof refers to telbivudine or a phosphate thereof. Telbivudine or a phosphate thereof is useful in the treatment and/or prevention of hepatitis B infection.

In one embodiment, a nucleoside or a phosphate thereof refers to zidovudine or a phosphate thereof. Zidovudine or a phosphate thereof is useful in the treatment and/or prevention of HIV/AIDS.

In one embodiment, a nucleoside or a phosphate thereof refers to idoxuridine or a phosphate thereof. Idoxuridine or a phosphate thereof is useful in the treatment and/or prevention of herpes simplex keratitis.

In one embodiment, a nucleoside or a phosphate thereof refers to trifluridine or a phosphate thereof. Trifluridine or a phosphate thereof is useful in the treatment and/or prevention of keratitis and keratoconjunctivitis caused by the herpes simplex virus types 1 and 2, and treatment of vaccinia virus infections of the eye.

In one embodiment, the nucleoside or a phosphate thereof refers to gemcitabine.

Gemcitabine, or 2′,2′-difluoro-2′-deoxycytidine (dFdC) (CAS ID: 95058-81-4), has the structure:

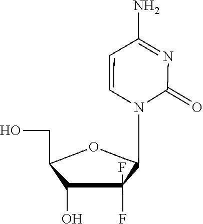

In one embodiment, the nucleoside or a phosphate thereof refers to a phosphate of gemcitabine.

In one embodiment, the nucleoside or a phosphate thereof refers to gemcitabine monophosphate.

Gemcitabine monophosphate, or 2′,2′-difluorodeoxycytidine 5′-phosphate (dFdCMP) (CAS ID: 116371-67-6), has the structure:

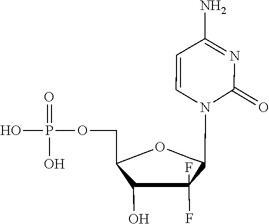

In one embodiment, the nucleoside or a phosphate thereof refers to gemcitabine diphosphate.

Gemcitabine diphosphate, or 2′,2′-difluorodeoxycytidine 5′-diphosphate (dFdCDP) (CAS ID: 116371-66-5), has the structure:

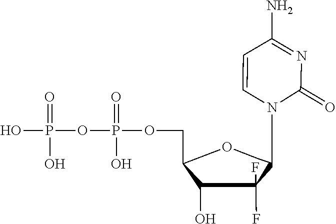

In one embodiment, the nucleoside or a phosphate thereof refers to gemcitabine triphosphate.

Gemcitabine triphosphate, or 2′,2′-difluorodeoxycytidine 5′-triphosphate (dFdCTP) (CAS ID: 110988-86-8), has the structure:

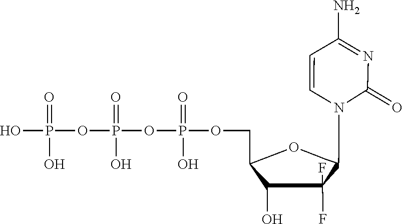

### Or a Phosphate Thereof

A phosphate is an anion, salt, functional group or ester derived from a phosphoric acid.

In one embodiment the phosphate is a derivative of orthophosphoric acid H₃PO₄.

In one embodiment the phosphate comprises a (PO₄)³⁻ group.

In one embodiment the phosphate comprises a P(O)(OH)₂O— group.

In one embodiment the phosphate comprises a diphosphate, for example a P(O)(OH)₂—O—P(O)(OH)—O— group.

In one embodiment the phosphate comprises a triphosphate, for example a P(O)(OH)₂—O—P(O)(OH)—O—P(O)(OH)— group.

In one embodiment the nucleoside or a phosphate thereof, is a monomeric unit, not polymerised into a longer chain.

In one embodiment the nucleoside or a phosphate thereof is not RNA or DNA.

### Amphipathic Cell Penetrating Peptide

In accordance with convention, the peptides described herein are drawn “N-terminus” first, i.e. on the left hand side.

In one embodiment an amphipathic cell penetrating peptide comprises or consists of the amino acid sequence WEARLARALARALARHLARALARALRACEA (SEQ ID_No 1) or a sequence with at least 80% sequence identity or homology.

In one embodiment an amphipathic cell penetrating peptide comprises the amino acid sequence WEARLARALARALARHLARALARALRACEA (SEQ ID_No 1) or a sequence with at least 80% sequence identity or homology.

In one embodiment an amphipathic cell penetrating peptide consists of the amino acid sequence WEARLARALARALARHLARALARALRACEA (SEQ ID_No 1) or a sequence with at least 80% sequence identity or homology.

In one embodiment an amphipathic cell penetrating peptide comprises or consists of the amino acid sequence WEARLARALARALARHLARALARALRACEA (SEQ ID_No 1) or a sequence with at least 85% sequence identity or homology.

In one embodiment an amphipathic cell penetrating peptide comprises the amino acid sequence WEARLARALARALARHLARALARALRACEA (SEQ ID_No 1) or a sequence with at least 85% sequence identity or homology.

In one embodiment an amphipathic cell penetrating peptide consists of the amino acid sequence WEARLARALARALARHLARALARALRACEA (SEQ ID_No 1) or a sequence with at least 85% sequence identity or homology.

In one embodiment an amphipathic cell penetrating peptide comprises or consists of the amino acid sequence WEARLARALARALARHLARALARALRACEA (SEQ ID_No 1) or a sequence with at least 90% sequence identity or homology.

In one embodiment an amphipathic cell penetrating peptide comprises the amino acid sequence WEARLARALARALARHLARALARALRACEA (SEQ ID_No 1) or a sequence with at least 90% sequence identity or homology.

In one embodiment an amphipathic cell penetrating peptide consists of the amino acid sequence WEARLARALARALARHLARALARALRACEA (SEQ ID_No 1) or a sequence with at least 90% sequence identity or homology.

In one embodiment an amphipathic cell penetrating peptide comprises or consists of the amino acid sequence WEARLARALARALARHLARALARALRACEA (SEQ ID_No 1) or a sequence with at least 95% sequence identity or homology.

In one embodiment an amphipathic cell penetrating peptide comprises the amino acid sequence WEARLARALARALARHLARALARALRACEA (SEQ ID_No 1) or a sequence with at least 95% sequence identity or homology.

In one embodiment an amphipathic cell penetrating peptide consists of the amino acid sequence WEARLARALARALARHLARALARALRACEA (SEQ ID_No 1) or a sequence with at least 95% sequence identity or homology.

In one embodiment an amphipathic cell penetrating peptide comprises or consists of the amino acid sequence WEARLARALARALARHLARALARALRACEA (SEQ ID_No 1).

In one embodiment an amphipathic cell penetrating peptide comprises the amino acid sequence WEARLARALARALARHLARALARALRACEA (SEQ ID_No 1).

In one embodiment an amphipathic cell penetrating peptide consists of the amino acid sequence WEARLARALARALARHLARALARALRACEA (SEQ ID_No 1).

In one embodiment a sequence with at least 80% sequence identity or homology to WEARLARALARALARHLARALARALRACEA (SEQ ID_No 1) consists of less than or equal to 35 amino acid residues.

In one embodiment a sequence with at least 80% sequence identity or homology to WEARLARALARALARHLARALARALRACEA (SEQ ID_No 1) consists of less than or equal to 30 amino acid residues.

In one embodiment a sequence with at least 80% sequence identity or homology to WEARLARALARALARHLARALARALRACEA (SEQ ID_No 1) consists of 26-30 amino acid residues.

In one embodiment a sequence with at least 80% sequence identity or homology to WEARLARALARALARHLARALARALRACEA (SEQ ID_No 1) comprises at least 5 arginine residues (R).

In one embodiment a sequence with at least 80% sequence identity or homology to WEARLARALARALARHLARALARALRACEA (SEQ ID_No 1) comprises at least 6 arginine residues (R).

In one embodiment a sequence with at least 80% sequence identity or homology to WEARLARALARALARHLARALARALRACEA (SEQ ID_No 1) comprises at least 10 Alanine Residues (A).

In one embodiment a sequence with at least 80% sequence identity or homology to WEARLARALARALARHLARALARALRACEA (SEQ ID_No 1) comprises at least 12 Alanine Residues (A).

In one embodiment a sequence with at least 80% sequence identity or homology to WEARLARALARALARHLARALARALRACEA (SEQ ID_No 1) comprises at least 5 leucine residues (L).

In one embodiment a sequence with at least 80% sequence identity or homology to WEARLARALARALARHLARALARALRACEA (SEQ ID_No 1) comprises at least 6 leucine residues (L).

In one embodiment a sequence with at least 80% sequence identity or homology to WEARLARALARALARHLARALARALRACEA (SEQ ID_No 1) comprises at least one cysteine residue (C).

In one embodiment a sequence with at least 80% sequence identity or homology to WEARLARALARALARHLARALARALRACEA (SEQ ID_No 1) comprises at least two but no more than three glutamic acid (E) residues.

In one embodiment a sequence with at least 80% sequence identity or homology to WEARLARALARALARHLARALARALRACEA (SEQ ID_No 1) comprises at least 6 arginine residues (R), at least 12 Alanine Residues (A), at least 6 leucine residues (L), optionally at least one cysteine residue (C) and at least two but no more than three glutamic acids residues (E).

In one embodiment a sequence with at least 80% sequence identity or homology to WEARLARALARALARHLARALARALRACEA (SEQ ID_No 1) comprises the consensus sequences EARLARALARALAR and/or LARALARALRA.

In one embodiment a sequence with at least 80% sequence identity or homology to WEARLARALARALARHLARALARALRACEA (SEQ ID_No 1) comprises the consensus sequence EARLARALARALAR.

In one embodiment a sequence with at least 80% sequence identity or homology to WEARLARALARALARHLARALARALRACEA (SEQ ID_No 1) comprises the consensus sequence LARALARALRA.

In one embodiment a sequence with at least 80% sequence identity or homology to WEARLARALARALARHLARALARALRACEA (SEQ ID_No 1) comprises the consensus sequences EARLARALARALAR and LARALARALRA.

In one embodiment a sequence with at least 80% sequence identity or homology to WEARLARALARALARHLARALARALRACEA (SEQ ID_No 1) does not comprise glycine (G).

A Sequence with at Least 80% Sequence Identity or Homology

Any of a variety of sequence alignment methods can be used to determine percent sequence identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Conventional methods include Altschul et al., Bull. Math. Bio. 48: 603-16, 1986 and Henikoff and Henikoff, Proc. Natl. Acad. Sci. USA 89:10915-19, 1992 where two amino acid sequences are aligned to optimize the alignment scores using a gap opening penalty of 10, a gap extension penalty of 1, and the “blosum 62” scoring matrix of Henikoff and Henikoff.

The percent sequence identity between two or more amino acid sequences is a function of the number of identical positions shared by the sequences. Thus, % identity may be calculated as the number of identical amino acids divided by the total number of amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Homologous sequences may be characterized as having one or more amino acid substitutions, deletions or additions. These changes are of a minor nature that do not significantly affect the folding or activity of the peptide. These may be small amino acid substitutions; small deletions; and small amino- or carboxyl-terminal extensions or other small additions.

In one embodiment the term “sequence identity or homology” refers to sequence identity.

In one embodiment the term “sequence identity or homology” refers to sequence homology.

### Nanoparticles

In one embodiment the nucleoside formulations described herein comprise or consist of nanoparticles. Nanoparticles may be formed by self-assembly by adding the nucleoside, or a phosphate thereof, and the amphipathic cell penetrating peptide together in ultrapure water.

In one embodiment there is provided a nanoparticle formulation comprising:
(i) a nucleoside or a phosphate thereof; and
(ii) an amphipathic cell penetrating peptide comprising or consisting of the amino acid sequence:

(SEQ ID_No 1)
WEARLARALARALARHLARALARALRACEA

or a sequence with at least 80% sequence identity or homology.

In one embodiment there is provided a nanoparticle formulation comprising:
(i) a gemcitabine or a phosphate thereof; and
(ii) an amphipathic cell penetrating peptide comprising or consisting of the amino acid sequence:

(SEQ ID_No 1)
WEARLARALARALARHLARALARALRACEA

or a sequence with at least 80% sequence identity or homology.

In one embodiment there is provided a nanoparticle formulation comprising:
(i) gemcitabine triphosphate; and
(ii) an amphipathic cell penetrating peptide comprising the amino acid sequence:

(SEQ ID_No 1)
WEARLARALARALARHLARALARALRACEA.

In one embodiment there is provided a nucleoside formulation as described herein wherein the formulation is a nanoparticle formulation.

In one embodiment there is provided a nanoparticle comprising a nucleoside formulation as described herein.

In one embodiment there is provided a nanoparticle formulation comprising a nucleoside formulation as described herein.

In one embodiment there is provided a nucleoside formulation as described herein, comprising nanoparticles.

In one embodiment there is provided a nucleoside formulation as described herein, comprising nanoparticles with a Z-Average of 30-150 nm. The Z average is the intensity weighted mean hydrodynamic size of the ensemble collection of particles measured by dynamic light scattering (DLS).

In one embodiment there is provided a nucleoside formulation as described herein, comprising nanoparticles with a Z-Average of 60-100 nm.

In one embodiment there is provided a nucleoside formulation as described herein, comprising nanoparticles with a polydispersity index of =<0.3. The polydispersity index (PI) is a measure of the heterogeneity of a sample based on size.

In one embodiment there is provided a nucleoside formulation as described herein, comprising nanoparticles with a polydispersity index of =<0.5.

### Mole Ratio of Nucleoside or a Phosphate Thereof:Amphipathic Cell Penetrating Peptide

In one embodiment, the mole ratio (mole:mole) of the nuceloside or a phosphate thereof:amphipathic cell penetrating peptide as described herein may be varied. This may have a beneficial effect on the physiochemical characteristics (for example the Z-Average, zeta potential (particle charge), and/or polydispersity index), the cellular uptake, and/or the treatment efficacy.

In one embodiment the mole ratio of nucleoside or a phosphate thereof:amphipathic cell penetrating peptide is about 0.1:10.

In one embodiment the mole ratio of nucleoside or a phosphate thereof:amphipathic cell penetrating peptide is 0.1:10.

In one embodiment the mole ratio of nucleoside or a phosphate thereof:amphipathic cell penetrating peptide is about 1:5.

In one embodiment the mole ratio of nucleoside or a phosphate thereof:amphipathic cell penetrating peptide is 1:5.

In one embodiment the mole ratio of nucleoside or a phosphate thereof:amphipathic cell penetrating peptide is about 1:1-3.

In one embodiment the mole ratio of nucleoside or a phosphate thereof:amphipathic cell penetrating peptide is 1:1-3.

In one embodiment the mole ratio of nucleoside or a phosphate thereof:amphipathic cell penetrating peptide is about 1:1.2-2.8.

In one embodiment the mole ratio of nucleoside or a phosphate thereof:amphipathic cell penetrating peptide is 1:1.2-2.8.

In one embodiment the mole ratio of nucleoside or a phosphate thereof:amphipathic cell penetrating peptide is about 1:1.4-2.6.

In one embodiment the mole ratio of nucleoside or a phosphate thereof:amphipathic cell penetrating peptide is 1:1.4-2.6.

In one embodiment the mole ratio of nucleoside or a phosphate thereof:amphipathic cell penetrating peptide is about 1:1.6-2.4.

In one embodiment the mole ratio of nucleoside or a phosphate thereof:amphipathic cell penetrating peptide is 1:1.6-2.4.

In one embodiment the mole ratio of nucleoside or a phosphate thereof:amphipathic cell penetrating peptide is about 1:1.8-2.2.

In one embodiment the mole ratio of nucleoside or a phosphate thereof:amphipathic cell penetrating peptide is 1:1.8-2.2.

In one embodiment the mole ratio of nucleoside or a phosphate thereof:amphipathic cell penetrating peptide is about 1:1.9-2.5.

In one embodiment the mole ratio of nucleoside or a phosphate thereof:amphipathic cell penetrating peptide is 1:1.9-2.5.

In one embodiment the mole ratio of nucleoside or a phosphate thereof:amphipathic cell penetrating peptide is about 1:2.0-2.4.

In one embodiment the mole ratio of nucleoside or a phosphate thereof:amphipathic cell penetrating peptide is 1:2.0-2.4.

In one embodiment the mole ratio of nucleoside or a phosphate thereof:amphipathic cell penetrating peptide is about 1:1.

In one embodiment the mole ratio of nucleoside or a phosphate thereof:amphipathic cell penetrating peptide is 1:1.

In one embodiment the mole ratio of nucleoside or a phosphate thereof:amphipathic cell penetrating peptide is about 1:1.2.

In one embodiment the mole ratio of nucleoside or a phosphate thereof:amphipathic cell penetrating peptide is 1:1.2.

In one embodiment the mole ratio of nucleoside or a phosphate thereof:amphipathic cell penetrating peptide is about 1:1.4.

In one embodiment the mole ratio of nucleoside or a phosphate thereof:amphipathic cell penetrating peptide is 1:1.4.

In one embodiment the mole ratio of nucleoside or a phosphate thereof:amphipathic cell penetrating peptide is about 1:1.6.

In one embodiment the mole ratio of nucleoside or a phosphate thereof:amphipathic cell penetrating peptide is 1:1.6.

In one embodiment the mole ratio of nucleoside or a phosphate thereof:amphipathic cell penetrating peptide is about 1:1.8.

In one embodiment the mole ratio of nucleoside or a phosphate thereof:amphipathic cell penetrating peptide is 1:1.8.

In one embodiment the mole ratio of nucleoside or a phosphate thereof:amphipathic cell penetrating peptide is about 1:2.

In one embodiment the mole ratio of nucleoside or a phosphate thereof:amphipathic cell penetrating peptide is 1:2.

In one embodiment the mole ratio of nucleoside or a phosphate thereof:amphipathic cell penetrating peptide is about 1:2.2.

In one embodiment the mole ratio of nucleoside or a phosphate thereof:amphipathic cell penetrating peptide is 1:2.2.

In one embodiment the mole ratio of nucleoside or a phosphate thereof:amphipathic cell penetrating peptide is about 1:2.4.

In one embodiment the mole ratio of nucleoside or a phosphate thereof:amphipathic cell penetrating peptide is 1:2.4.

In one embodiment the mole ratio of nucleoside or a phosphate thereof:amphipathic cell penetrating peptide is about 1:2.6.

In one embodiment the mole ratio of nucleoside or a phosphate thereof:amphipathic cell penetrating peptide is 1:2.6.

In one embodiment the mole ratio of nucleoside or a phosphate thereof:amphipathic cell penetrating peptide is about 1:2.8.

In one embodiment the mole ratio of nucleoside or a phosphate thereof:amphipathic cell penetrating peptide is 1:2.8.

In one embodiment the mole ratio of nucleoside or a phosphate thereof:amphipathic cell penetrating peptide is about 1:3.

In one embodiment the mole ratio of nucleoside or a phosphate thereof:amphipathic cell penetrating peptide is 1:3.

In one embodiment the mole ratio of nucleoside or a phosphate thereof:amphipathic cell penetrating peptide is about 0.1:3.

In one embodiment the mole ratio of nucleoside or a phosphate thereof:amphipathic cell penetrating peptide is 0.1:3.

In one embodiment the mole ratio of nucleoside or a phosphate thereof:amphipathic cell penetrating peptide is about 1:4.

In one embodiment the mole ratio of nucleoside or a phosphate thereof:amphipathic cell penetrating peptide is 1:4.

In one embodiment the mole ratio of nucleoside or a phosphate thereof:amphipathic cell penetrating peptide is about 1:5.

In one embodiment the mole ratio of nucleoside or a phosphate thereof:amphipathic cell penetrating peptide is 1:5.

In one embodiment the mole ratio of gemcitabine or a phosphate thereof:amphipathic cell penetrating peptide is about 0.1:1-10.

In one embodiment the mole ratio of gemcitabine or a phosphate thereof:amphipathic cell penetrating peptide is 0.1:1-3.

In one embodiment the mole ratio of gemcitabine or a phosphate thereof:amphipathic cell penetrating peptide is about 1:1-5.

In one embodiment the mole ratio of gemcitabine or a phosphate thereof:amphipathic cell penetrating peptide is 1:1-5.

In one embodiment the mole ratio of gemcitabine or a phosphate thereof:amphipathic cell penetrating peptide is about 1:1-3.

In one embodiment the mole ratio of gemcitabine or a phosphate thereof:amphipathic cell penetrating peptide is 1:1-3.

In one embodiment the mole ratio of gemcitabine or a phosphate thereof:amphipathic cell penetrating peptide is about 1:1.2-2.8.

In one embodiment the mole ratio of gemcitabine or a phosphate thereof:amphipathic cell penetrating peptide is 1:1.2-2.8.

In one embodiment the mole ratio of gemcitabine or a phosphate thereof:amphipathic cell penetrating peptide is about 1:1.4-2.6.

In one embodiment the mole ratio of gemcitabine or a phosphate thereof:amphipathic cell penetrating peptide is 1:1.4-2.6.

In one embodiment the mole ratio of gemcitabine or a phosphate thereof:amphipathic cell penetrating peptide is about 1:1.6-2.4.

In one embodiment the mole ratio of gemcitabine or a phosphate thereof:amphipathic cell penetrating peptide is 1:1.6-2.4.

In one embodiment the mole ratio of gemcitabine or a phosphate thereof:amphipathic cell penetrating peptide is about 1:1.8-2.2.

In one embodiment the mole ratio of gemcitabine or a phosphate thereof:amphipathic cell penetrating peptide is 1:1.8-2.2.

In one embodiment the mole ratio of gemcitabine or a phosphate thereof:amphipathic cell penetrating peptide is about 1:2.0-2.4.

In one embodiment the mole ratio of gemcitabine or a phosphate thereof:amphipathic cell penetrating peptide is 1:2.0-2.4.

In one embodiment the mole ratio of gemcitabine or a phosphate thereof:amphipathic cell penetrating peptide is about 1:1.9-2.5.

In one embodiment the mole ratio of gemcitabine or a phosphate thereof:amphipathic cell penetrating peptide is 1:1.9-2.5.

In one embodiment the mole ratio of gemcitabine or a phosphate thereof:amphipathic cell penetrating peptide is about 1:1.

In one embodiment the mole ratio of gemcitabine or a phosphate thereof:amphipathic cell penetrating peptide is 1:1.

In one embodiment the mole ratio of gemcitabine or a phosphate thereof:amphipathic cell penetrating peptide is about 1:1.2.

In one embodiment the mole ratio of gemcitabine or a phosphate thereof:amphipathic cell penetrating peptide is 1:1.2.

In one embodiment the mole ratio of gemcitabine or a phosphate thereof:amphipathic cell penetrating peptide is about 1:1.4.

In one embodiment the mole ratio of gemcitabine or a phosphate thereof:amphipathic cell penetrating peptide is 1:1.4.

In one embodiment the mole ratio of gemcitabine or a phosphate thereof:amphipathic cell penetrating peptide is about 1:1.6.

In one embodiment the mole ratio of gemcitabine or a phosphate thereof:amphipathic cell penetrating peptide is 1:1.6.

In one embodiment the mole ratio of gemcitabine or a phosphate thereof:amphipathic cell penetrating peptide is about 1:1.8.

In one embodiment the mole ratio of gemcitabine or a phosphate thereof:amphipathic cell penetrating peptide is 1:1.8.

In one embodiment the mole ratio of gemcitabine or a phosphate thereof:amphipathic cell penetrating peptide is about 1:2.

In one embodiment the mole ratio of gemcitabine or a phosphate thereof:amphipathic cell penetrating peptide is 1:2.

In one embodiment the mole ratio of gemcitabine or a phosphate thereof:amphipathic cell penetrating peptide is about 1:2.2.

In one embodiment the mole ratio of gemcitabine or a phosphate thereof:amphipathic cell penetrating peptide is 1:2.2.

In one embodiment the mole ratio of gemcitabine or a phosphate thereof:amphipathic cell penetrating peptide is about 1:2.4.

In one embodiment the mole ratio of gemcitabine or a phosphate thereof:amphipathic cell penetrating peptide is 1:2.4.

In one embodiment the mole ratio of gemcitabine or a phosphate thereof:amphipathic cell penetrating peptide is about 1:2.6.

In one embodiment the mole ratio of gemcitabine or a phosphate thereof:amphipathic cell penetrating peptide is 1:2.6.

In one embodiment the mole ratio of gemcitabine or a phosphate thereof:amphipathic cell penetrating peptide is about 1:2.8.

In one embodiment the mole ratio of gemcitabine or a phosphate thereof:amphipathic cell penetrating peptide is 1:2.8.

In one embodiment the mole ratio of gemcitabine or a phosphate thereof:amphipathic cell penetrating peptide is about 1:3.

In one embodiment the mole ratio of gemcitabine or a phosphate thereof:amphipathic cell penetrating peptide is 1:3.

In one embodiment the mole ratio of gemcitabine or a phosphate thereof:amphipathic cell penetrating peptide is about 1:5.

In one embodiment the mole ratio of gemcitabine or a phosphate thereof:amphipathic cell penetrating peptide is 1:5.

In one embodiment the mole ratio of gemcitabine or a phosphate thereof:amphipathic cell penetrating peptide is about 0.1:10.

In one embodiment the mole ratio of gemcitabine or a phosphate thereof:amphipathic cell penetrating peptide is 0.1:10.

In one embodiment the mole ratio of gemcitabine triphosphate:amphipathic cell penetrating peptide is about 0.1:1-10.

In one embodiment the mole ratio of gemcitabine triphosphate:amphipathic cell penetrating peptide is 0.1:1-10.

In one embodiment the mole ratio of gemcitabine triphosphate:amphipathic cell penetrating peptide is about 1:1-5.

In one embodiment the mole ratio of gemcitabine triphosphate:amphipathic cell penetrating peptide is 1:1-5.

In one embodiment the mole ratio of gemcitabine triphosphate:amphipathic cell penetrating peptide is about 1:1-3.

In one embodiment the mole ratio of gemcitabine triphosphate:amphipathic cell penetrating peptide is 1:1-3.

In one embodiment the mole ratio of gemcitabine triphosphate:amphipathic cell penetrating peptide is about 1:1.2-2.8.

In one embodiment the mole ratio of gemcitabine triphosphate:amphipathic cell penetrating peptide is 1:1.2-2.8.

In one embodiment the mole ratio of gemcitabine triphosphate:amphipathic cell penetrating peptide is about 1:1.4-2.6.

In one embodiment the mole ratio of gemcitabine triphosphate:amphipathic cell penetrating peptide is 1:1.4-2.6.

In one embodiment the mole ratio of gemcitabine triphosphate:amphipathic cell penetrating peptide is about 1:1.6-2.4.

In one embodiment the mole ratio of gemcitabine triphosphate:amphipathic cell penetrating peptide is 1:1.6-2.4.

In one embodiment the mole ratio of gemcitabine triphosphate:amphipathic cell penetrating peptide is about 1:1.8-2.2.

In one embodiment the mole ratio of gemcitabine triphosphate:amphipathic cell penetrating peptide is 1:1.8-2.2.

In one embodiment the mole ratio of gemcitabine triphosphate:amphipathic cell penetrating peptide is about 1:2.0-2.4.

In one embodiment the mole ratio of gemcitabine triphosphate:amphipathic cell penetrating peptide is 1:2.0-2.4.

In one embodiment the mole ratio of gemcitabine triphosphate:amphipathic cell penetrating peptide is about 1:1.9-2.5.

In one embodiment the mole ratio of gemcitabine triphosphate:amphipathic cell penetrating peptide is 1:1.9-2.5.

In one embodiment the mole ratio of gemcitabine triphosphate:amphipathic cell penetrating peptide is about 1:1.

In one embodiment the mole ratio of gemcitabine triphosphate:amphipathic cell penetrating peptide is 1:1.

In one embodiment the mole ratio of gemcitabine triphosphate:amphipathic cell penetrating peptide is about 1:1.2.

In one embodiment the mole ratio of gemcitabine triphosphate:amphipathic cell penetrating peptide is 1:1.2.

In one embodiment the mole ratio of gemcitabine triphosphate:amphipathic cell penetrating peptide is about 1:1.4.

In one embodiment the mole ratio of gemcitabine triphosphate:amphipathic cell penetrating peptide is 1:1.4.

In one embodiment the mole ratio of gemcitabine triphosphate:amphipathic cell penetrating peptide is about 1:1.6.

In one embodiment the mole ratio of gemcitabine triphosphate:amphipathic cell penetrating peptide is 1:1.6.

In one embodiment the mole ratio of gemcitabine triphosphate:amphipathic cell penetrating peptide is about 1:1.8.

In one embodiment the mole ratio of gemcitabine triphosphate:amphipathic cell penetrating peptide is 1:1.8.

In one embodiment the mole ratio of gemcitabine triphosphate:amphipathic cell penetrating peptide is about 1:2.

In one embodiment the mole ratio of gemcitabine triphosphate:amphipathic cell penetrating peptide is 1:2.

In one embodiment the mole ratio of gemcitabine triphosphate:amphipathic cell penetrating peptide is about 1:2.2.

In one embodiment the mole ratio of gemcitabine triphosphate:amphipathic cell penetrating peptide is 1:2.2.

In one embodiment the mole ratio of gemcitabine triphosphate:amphipathic cell penetrating peptide is about 1:2.4.

In one embodiment the mole ratio of gemcitabine triphosphate:amphipathic cell penetrating peptide is 1:2.4.

In one embodiment the mole ratio of gemcitabine triphosphate:amphipathic cell penetrating peptide is about 1:2.6.

In one embodiment the mole ratio of gemcitabine triphosphate:amphipathic cell penetrating peptide is 1:2.6.

In one embodiment the mole ratio of gemcitabine triphosphate:amphipathic cell penetrating peptide is about 1:2.8.

In one embodiment the mole ratio of gemcitabine triphosphate:amphipathic cell penetrating peptide is 1:2.8.

In one embodiment the mole ratio of gemcitabine triphosphate:amphipathic cell penetrating peptide is 1:3.

In one embodiment the mole ratio of gemcitabine triphosphate:amphipathic cell penetrating peptide is about 1:3.

In one embodiment the mole ratio of gemcitabine triphosphate:amphipathic cell penetrating peptide is 1:5.

In one embodiment the mole ratio of gemcitabine triphosphate:amphipathic cell penetrating peptide is about 1:5.

In one embodiment the mole ratio of gemcitabine triphosphate:amphipathic cell penetrating peptide is 0.1:10.

In one embodiment the mole ratio of gemcitabine triphosphate:amphipathic cell penetrating peptide is about 0.1:10.

### Bulking Agents, Cryoprotectants and Agents Inferring Tonicity

In one embodiment, a bulking agent may be added prior to lyophilisation of nanoparticles for transport and storage. Bulking agents are additives that increase the bulk-volume of a product without affecting its properties.

In one embodiment, a cryoprotectant may be added prior to lyophilisation of nanoparticles. A cryoprotectant is a substance used to protect biological tissue from freezing damage.

In one embodiment, a solute may be added to infer tonicity, e.g. to produce an isotonic formulation once water is added to the formulation. An isotonic formulation possesses the same concentration of solutes as the blood, i.e. 290-310 mOsmol/kg.

In one embodiment, the osmolality of a solution of a nucleoside formulation described herein in water is 10-1000 mOsmol/kg.

In one embodiment, the osmolality of a solution of a nucleoside formulation described herein in water is 100-500 mOsmol/kg.

In one embodiment, the osmolality of a solution of a nucleoside formulation described herein in water is 200-400 mOsmol/kg.

In one embodiment, the osmolality of a solution of a nucleoside formulation described herein in water is 290-310 mOsmol/kg.

In one embodiment, the osmolality of a solution of a nucleoside formulation described herein in water is about 300 mOsmol/kg.

In one embodiment, the osmolality of a solution of a nucleoside formulation described herein in water is 300 mOsmol/kg.

Suitable bulking agents include trehalose, sucrose, mannose, dextrose or any mixture of such agents. These agents may also be employed as cryoprotectants and/or agents to infer tonicity.

In one embodiment, the nucleoside formulations described herein, additionally comprise trehalose, sucrose, mannose, dextrose or any mixture of such agents.

In one embodiment, the nucleoside formulations described herein, additionally comprise >85% w/w trehalose, sucrose, mannose, dextrose or any mixture of such agents.

In one embodiment, the nucleoside formulations described herein, additionally comprise >90% w/w trehalose, sucrose, mannose, dextrose or any mixture of such agents.

In one embodiment, the nucleoside formulations described herein, additionally comprise >95% w/w trehalose, sucrose, mannose, dextrose or any mixture of such agents.

In one embodiment the bulking agent is trehalose.

In one embodiment nucleoside formulations described herein, additionally comprise trehalose.

In one embodiment nucleoside formulations described herein, additionally comprise >85% w/w trehalose.

In one embodiment nucleoside formulations described herein, additionally comprise >90% w/w trehalose.

In one embodiment nucleoside formulations described herein, additionally comprise >95% w/w trehalose.

In one embodiment the bulking agent is sucrose.

In one embodiment nucleoside formulations described herein, additionally comprise sucrose.

In one embodiment nucleoside formulations described herein, additionally comprise >85% w/w sucrose.

In one embodiment nucleoside formulations described herein, additionally comprise >90% w/w sucrose.

In one embodiment nucleoside formulations described herein, additionally comprise >95% w/w sucrose.

In one embodiment the bulking agent is mannose.

In one embodiment nucleoside formulations described herein, additionally comprise mannose.

In one embodiment nucleoside formulations described herein, additionally comprise >85% w/w mannose.

In one embodiment nucleoside formulations described herein, additionally comprise >90% w/w mannose.

In one embodiment nucleoside formulations described herein, additionally comprise >95% w/w mannose.

In one embodiment the bulking agent comprises trehalose.

In one embodiment the bulking agent comprises sucrose.

In one embodiment the bulking agent comprises mannose.

In one embodiment the bulking agent comprises dextrose.

In one embodiment the nucleoside formulation as described herein comprises:
(i) 0.1-1% w/w gemcitabine or a phosphate thereof;
(ii) 1-10% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 89-99% w/w trehalose, sucrose, mannose and/or dextrose.

In one embodiment the nucleoside formulation as described herein comprises:
(i) 0.2-0.5% w/w gemcitabine or a phosphate thereof;
(ii) 3.5-4.5% w/w an amphipathic cell penetrating peptide consisting of the amino acid sequence WEARLARALARALARHLARALARALRACEA (SEQ ID_No 1) or a sequence with at least 80% sequence identity or homology; and
(iii) 95-97% w/w trehalose, sucrose, mannose and/or dextrose.

In one embodiment the nucleoside formulation as described herein comprises:
(i) 0.1-1% w/w gemcitabine triphosphate;
(ii) 1-10% w/w an amphipathic cell penetrating peptide consisting of the amino acid sequence WEARLARALARALARHLARALARALRACEA (SEQ ID_No 1); and
(iii) 89-99% w/w trehalose.

In one embodiment the nucleoside formulation as described herein comprises:
(i) 0.2-0.5% w/w gemcitabine triphosphate;
(ii) 3.5-4.5% w/w an amphipathic cell penetrating peptide consisting of the amino acid sequence WEARLARALARALARHLARALARALRACEA (SEQ ID_No 1); and
(iii) 95-97% w/w trehalose.

In one embodiment the nucleoside formulation as described herein comprises:
(i) about 0.3% w/w gemcitabine triphosphate;
(ii) about 4% w/w an amphipathic cell penetrating peptide consisting of the amino acid sequence WEARLARALARALARHLARALARALRACEA (SEQ ID_No 1); and
(iii) about 96% w/w trehalose.

In one embodiment the nucleoside formulation as described herein comprises:
(i) 0.3% w/w gemcitabine triphosphate;
(ii) 4.03% w/w an amphipathic cell penetrating peptide consisting of the amino acid sequence WEARLARALARALARHLARALARALRACEA (SEQ ID_No 1); and
(iii) 95.67% w/w trehalose.

### Formulations in Water

The nucleoside formulations described herein may be conveniently formulated in water, particularly ultrapure water, for ease of administration, particularly via intravenous injection.

A nucleoside formulation as described herein comprising:
(i) 0.1-1% w/w gemcitabine or a phosphate thereof;
(ii) 1-10% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 89-99% w/w trehalose, sucrose, mannose and/or dextrose;
   formulated as an isotonic formulation in water with an osmolality of 290-310 mOsmol/kg.

A nucleoside formulation as described herein comprising:
(i) 0.2-0.5% w/w gemcitabine or a phosphate thereof;
(ii) 3.5-4.5% w/w an amphipathic cell penetrating peptide consisting of the amino acid sequence WEARLARALARALARHLARALARALRACEA (SEQ ID_No 1) or a sequence with at least 80% sequence identity or homology; and
(iii) 95-97% w/w trehalose, sucrose, mannose and/or dextrose;
   formulated as an isotonic formulation in water with an osmolality of 290-310 mOsmol/kg.

A nucleoside formulation as described herein comprising:
(i) 0.1-1% w/w gemcitabine triphosphate;
(ii) 1-10% w/w an amphipathic cell penetrating peptide consisting of the amino acid sequence WEARLARALARALARHLARALARALRACEA (SEQ ID_No 1); and
(iii) 89-99% w/w trehalose;
   formulated as an isotonic formulation in water with an osmolality of 290-310 mOsmol/kg.

A nucleoside formulation as described herein comprising:
(i) 0.2-0.5% w/w gemcitabine triphosphate;
(ii) 3.5-4.5% w/w an amphipathic cell penetrating peptide consisting of the amino acid sequence WEARLARALARALARHLARALARALRACEA (SEQ ID_No 1); and
(iii) 95-97% w/w trehalose;
   formulated as an isotonic formulation in water with an osmolality of 290-310 mOsmol/kg.

A nucleoside formulation as described herein comprising:
(i) about 0.3% w/w gemcitabine triphosphate;
(ii) about 4% w/w an amphipathic cell penetrating peptide consisting of the amino acid sequence WEARLARALARALARHLARALARALRACEA (SEQ ID_No 1); and
(iii) about 96% w/w trehalose;
   formulated as an isotonic formulation in water with an osmolality of 290-310 mOsmol/kg.

A nucleoside formulation as described herein comprising:
(i) 0.3% w/w gemcitabine triphosphate;
(ii) 4.03% w/w an amphipathic cell penetrating peptide consisting of the amino acid sequence WEARLARALARALARHLARALARALRACEA (SEQ ID_No 1); and
(iii) 95.67% w/w trehalose;
   formulated as an isotonic formulation in water with an osmolality of 290-310 mOsmol/kg.

### Automated Formulation

In one embodiment, the nanoparticles are prepared via an automated controllable mixing system, for example an automated microfluidics system, for example Precision Nanosystems Ignite NanoAssemblr. This technology has the potential to control both the mixing rate and the mixing ratios during formulation of the nanoparticles, resulting in a reduction in Z-average particle size, resulting in an additional decrease in the polydispersity index when compared to manual formulation methods.

Microfluidics refers to the behaviour, precise control, and manipulation of fluids that are geometrically constrained to a small scale (typically sub-millimeter) at which surface forces dominate volumetric forces.

In one embodiment the nanoparticles as described herein are prepared via an automated controlled mixing system.

In one embodiment there is provided a method of preparing a nanoparticle formulation which comprises formulating (i) a solution of WEARLARALARALARHLARALARALRACEA (SEQ ID_No 1) or a sequence with at least 80% sequence identity or homology with (ii) a pharmacologically active agent, in an automated controlled mixing system, particularly an automated microfluidics system.

In one embodiment there is provided a method of preparing a nanoparticle formulation which comprises formulating (i) a solution of WEARLARALARALARHLARALARALRACEA (SEQ ID_No 1) or a sequence with at least 80% sequence identity or homology with (ii) a nucleic acid or other agent, wherein the other agent is preferably a negatively charged or hydrophilic compound, in an automated controlled mixing system, particularly an automated microfluidics system.

In one embodiment there is provided a method of preparing a nanoparticle formulation which comprises formulating (i) a solution of WEARLARALARALARHLARALARALRACEA (SEQ ID_No 1) or a sequence with at least 80% sequence identity or homology and (ii) DNA, RNA, shRNA, and siRNA, in an automated controlled mixing system, particularly an automated microfluidics system.

In one embodiment there is provided a method of preparing a nanoparticle formulation which comprises (i) formulating a solution of WEARLARALARALARHLARALARALRACEA (SEQ ID_No 1) or a sequence with at least 80% sequence identity or homology with (ii) a bisphophonate drug including alendronate, etidronate, zolendrate or any other nitrogen or non-nitrogen based bisphosphonate drug, in an automated controlled mixing system, particularly an automated microfluidics system.

In one embodiment there is provided a method of preparing a nanoparticle formulation which comprises formulating (i) a solution of WEARLARALARALARHLARALARALRACEA (SEQ ID_No 1) or a sequence with at least 80% sequence identity or homology and (ii) gold, in an automated controlled mixing system, particularly an automated microfluidics system.

In one embodiment there is provided a method of preparing a nanoparticle formulation which comprises (i) formulating a solution of WEARLARALARALARHLARALARALRACEA (SEQ ID_No 1) or a sequence with at least 80% sequence identity or homology with (ii) a nucleoside or a phosphate thereof, in an automated controlled mixing system, particularly an automated microfluidics system.

In one embodiment there is provided a method of preparing a nanoparticle formulation which comprises (i) formulating a solution of WEARLARALARALARHLARALARALRACEA (SEQ ID_No 1) or a sequence with at least 80% sequence identity or homology and (ii) gemcitabine or a phosphate thereof, in an automated controlled mixing system, particularly an automated microfluidics system.

In one embodiment there is provided a method of preparing a nanoparticle formulation which comprises (i) formulating a solution of WEARLARALARALARHLARALARALRACEA (SEQ ID_No 1) or a sequence with at least 80% sequence identity or homology and (ii) gemcitabine triphosphate, in an automated controlled mixing system, particularly an automated microfluidics system.

In one embodiment, the flow rate ratio of the amphipathic peptide described herein pharmacologically active agent of the automated microfluidics system is 1:1.

In one embodiment, the flow rate ratio of the amphipathic peptide described herein pharmacologically active agent of the automated microfluidics system is about 1:1.

### Formulation

Nanoparticles may be formed by self-assembly by adding the nucleoside or a phosphate thereof and the amphipathic cell penetrating peptide together in ultrapure water with instantaneous formulation occurring. The resulting nucleoside formulation may be lyophilised for transport and storage, and then rehydrate in water for use.

The nucleoside formulation described herein may be employed in various routes of administration, for example oral, nasal, rectal, topically, percutaneous, intravitreal, intravenous, or intramuscular, intradermal administration, particularly intravenous administration. In one embodiment, the nucleoside formulation of the present disclosure may be employed in an injectable formulation, for example an intravenous injection.

### Charge Density

The mean surface charge density of nanoparticles may be a contributing factor to their toxicity by promoting oxidative stress mechanisms which in turn can promote mitochondrial dysfunction and viability loss.

The charge density may be measured by polyelectrolytic titration using methods described in Ritz et al, Biomacromolecules. 2015 Apr. 13; 16(4):1311-21. doi: 10.1021/acs.biomac.5b00108. Epub 2015 Apr. 3 and Weiss et al, J Nanobiotechnology. 2021 Jan. 6; 19(1):5. doi: 10.1186/s12951-020-00747-7.

Polyelectrolytic titration may be performed using poly(acrylic acid) (PAA) 0.01 M at pH 7.4 and addition of PAA to nanoparticles and measuring the charge creates a sigmoidal curve of which the volume (V) can be derived from the equivalence point. In conjunction, the mass of peptide in the system (w) and concentration of PAA (0.01 M) used, is used to calculate the average charge density of the nanoparticle (Qₑₖ).

In one embodiment, the mean surface charge density of the nanoparticles comprising gemcitabine or a phosphate thereof, and the amphipathic cell penetrating peptide as described herein is ≤2 μmol/mg at 20° C. Mean surface charge density figures were measured using an Orion STAR® multi parameter bench meter.

In one embodiment, the mean surface charge density of the nanoparticles comprising gemcitabine or a phosphate thereof, and the amphipathic cell penetrating peptide as described herein is ≤1.5 μmol/mg at 20° C.

In one embodiment, the mean surface charge density of the nanoparticles comprising gemcitabine or a phosphate thereof, and the amphipathic cell penetrating peptide as described herein is about 1 μmol/mg at 20° C.

In one embodiment, the mean surface charge density of the nanoparticles comprising gemcitabine or a phosphate thereof, and the amphipathic cell penetrating peptide as described herein is 1 μmol/mg at 20° C.

### Uses

In one embodiment there is provided a nucleoside formulation as described herein for use as a medicament.

In one embodiment there is provided the use of a nucleoside formulation as described herein as a medicament.

In one embodiment there is provided a nucleoside formulation as described herein for use in therapy.

In one embodiment there is provided a nucleoside formulation as described herein for use in an intravenous injection.

In one embodiment there is provided a nucleoside formulation as described herein for use in the treatment of viral infections.

In one embodiment there is provided a nucleoside formulation as described herein for use in the treatment of HIV, ebola, Marburg, coronavirus, hepatitis B, hepatitis C, vaccinia, Epstein-Barr, cytomegalovirus, zikia, herpes simplex and/or varicella zoster virus infection.

In one embodiment there is provided a nucleoside formulation as described herein for use in the treatment of cancer.

As used herein, the terms “treatment” and “treat” refer to reversing, alleviating, delaying the onset of, or inhibiting the progress of a disease or disorder, or one or more symptoms thereof, as described herein. In some embodiments, treatment may be conducted after one or more symptoms have developed. In other embodiments, treatment may be conducted in the absence of symptoms. For example, treatment may be conducted to a susceptible individual prior to the onset of symptoms (e.g. in light of a history of symptoms and/or in light of genetic or other susceptibility factors). Treatment may also be continued after symptoms have resolved, for example to present or delay their recurrence.

Herein where use in the treatment of cancer is described, this may be cancer in early stage, actively progressing, metastatic and/or drug-resistant cancer. In some embodiments where cancer is referred to, the cancer is early cancer. In some embodiments where cancer is referred to, the cancer is locally advanced cancer. In some embodiments where cancer is referred to, the cancer is locally advanced and/or metastatic cancer. In some embodiments where cancer is referred to, the cancer is metastatic cancer. In some embodiments where cancer is referred to the cancer is invasive cancer.

In one embodiment there is provided a nucleoside formulation as described herein for use in the treatment of breast, bladder, testicular, pancreatic, ovarian or non-small cell lung cancer.

In one embodiment there is provided a nucleoside formulation as described herein for use in the treatment of breast cancer.

In one embodiment there is provided a nucleoside formulation as described herein for use in the treatment of testicular cancer.

In one embodiment there is provided a nucleoside formulation as described herein for use in the treatment of bladder cancer.

In one embodiment there is provided a nucleoside formulation as described herein for use in the treatment of pancreatic cancer.

In one embodiment there is provided a nucleoside formulation as described herein for use in the treatment of ovarian cancer.

In one embodiment there is provided a nucleoside formulation as described herein for use in the treatment of non-small cell lung cancer.

In one embodiment there is provided a nucleoside formulation as described herein for use in the treatment of metastatic breast cancer.

In one embodiment there is provided a nucleoside formulation as described herein for use in the first-line treatment of metastatic breast cancer.

In one embodiment there is provided a nucleoside formulation as described herein for use in the first-line treatment of metastatic breast cancer after failure of adjuvant chemotherapy.

In one embodiment there is provided a nucleoside formulation as described herein for use in the first-line treatment of patients with inoperable, locally advanced (Stage IIIA or IIIB), or metastatic (Stage IV) non-small cell lung cancer.

In one embodiment there is provided a nucleoside formulation as described herein for use in the treatment of locally advanced (nonresectable Stage II or Stage Ill) or metastatic (Stage IV) adenocarcinoma of the pancreas.

In one embodiment there is provided a nucleoside formulation as described herein for use in the treatment of locally advanced or metastatic bladder cancer.

In one embodiment there is provided a nucleoside formulation as described herein for use in the treatment of locally advanced or metastatic epithelial ovarian carcinoma.

### Pharmaceutical Compositions

In one embodiment there is provided a pharmaceutical composition which comprises a nucleoside formulation as described herein for use in an intravenous injection.

In one embodiment there is provided a pharmaceutical composition which comprises a nucleoside formulation as described herein for use in the treatment of viral infections.

In one embodiment there is provided a pharmaceutical composition which comprises a nucleoside formulation as described herein for use in the treatment of HIV, ebola, Marburg, coronavirus, hepatitis B, hepatitis C, vaccinia, Epstein-Barr, cytomegalovirus, zikia, herpes simplex and/or varicella zoster virus infection.

In one embodiment there is provided a pharmaceutical composition which comprises a nucleoside formulation as described herein for use in the treatment of cancer.

In one embodiment there is provided a pharmaceutical composition which comprises a nucleoside formulation as described herein for use in the treatment of breast, bladder, testicular, pancreatic, ovarian or non-small cell lung cancer.

In one embodiment there is provided a pharmaceutical composition which comprises a nucleoside formulation as described herein for use in the treatment of breast cancer.

In one embodiment there is provided a pharmaceutical composition which comprises a nucleoside formulation as described herein for use in the treatment of testicular cancer.

In one embodiment there is provided a pharmaceutical composition which comprises a nucleoside formulation as described herein for use in the treatment of bladder cancer.

In one embodiment there is provided a pharmaceutical composition which comprises a nucleoside formulation as described herein for use in the treatment of pancreatic cancer.

In one embodiment there is provided a pharmaceutical composition which comprises a nucleoside formulation as described herein for use in the treatment of ovarian cancer.

In one embodiment there is provided a pharmaceutical composition which comprises a nucleoside formulation as described herein for use in the treatment of non-small cell lung cancer.

In one embodiment there is provided a pharmaceutical composition which comprises a nucleoside formulation as described herein for use in the treatment of metastatic breast cancer.

In one embodiment there is provided a pharmaceutical composition which comprises a nucleoside formulation as described herein for use in the first-line treatment of metastatic breast cancer.

In one embodiment there is provided a pharmaceutical composition which comprises a nucleoside formulation as described herein for use in the first-line treatment of metastatic breast cancer after failure of adjuvant chemotherapy.

In one embodiment there is provided a pharmaceutical composition which comprises a nucleoside formulation as described herein for use in the first-line treatment of patients with inoperable, locally advanced (Stage IIIA or IIIB), or metastatic (Stage IV) non-small cell lung cancer.

In one embodiment there is provided a pharmaceutical composition which comprises a nucleoside formulation as described herein for use in the treatment of locally advanced (nonresectable Stage II or Stage III) or metastatic (Stage IV) adenocarcinoma of the pancreas.

In one embodiment there is provided a pharmaceutical composition which comprises a nucleoside formulation as described herein for use in the treatment of locally advanced or metastatic bladder cancer.

In one embodiment there is provided a pharmaceutical composition which comprises a nucleoside formulation as described herein for use in the treatment of locally advanced or metastatic epithelial ovarian carcinoma.

### Methods of Treatment

In one embodiment there is provided a method of intravenous injection which comprises administering a nucleoside formulation as described herein.

In one embodiment there is provided a method of treating viral infections which comprises administering to said animal an effective amount of a nucleoside formulation as described herein.

In one embodiment there is provided a method of treating HIV, ebola, Marburg, coronavirus, hepatitis B, hepatitis C, vaccinia, Epstein-Barr, cytomegalovirus, zikia, herpes simplex and/or varicella zoster virus infection which comprises administering to said animal an effective amount of a nucleoside formulation as described herein.

In one embodiment there is provided a method of treating cancer in a warm-blooded animal, such as man, which comprises administering to said animal an effective amount of a nucleoside formulation as described herein.

In one embodiment there is provided a method of treating breast, bladder, testicular, pancreatic, ovarian or non-small cell lung cancer in a warm-blooded animal, such as man, which is comprises administering to said animal an effective amount of a nucleoside formulation as described herein.

In one embodiment there is provided a method of treating breast cancer in a warm-blooded animal, such as man, which comprises administering to said animal an effective amount of a nucleoside formulation as described herein.

In one embodiment there is provided a method of treating testicular cancer in a warm-blooded animal, such as man, which comprises administering to said animal an effective amount of a nucleoside formulation as described herein.

In one embodiment there is provided a method of treating bladder cancer in a warm-blooded animal, such as man, which comprises administering to said animal an effective amount of a nucleoside formulation as described herein.

In one embodiment there is provided a method of treating pancreatic cancer in a warm-blooded animal, such as man, which comprises administering to said animal an effective amount of a nucleoside formulation as described herein.

In one embodiment there is provided a method of treating ovarian cancer in a warm-blooded animal, such as man, which comprises administering to said animal an effective amount of a nucleoside formulation as described herein.

In one embodiment there is provided a method of treating non-small cell lung cancer in a warm-blooded animal, such as man, which comprises administering to said animal an effective amount of a nucleoside formulation as described herein.

In one embodiment there is provided a method of treating metastatic breast cancer in a warm-blooded animal, such as man, which comprises administering to said animal an effective amount of a nucleoside formulation as described herein.

In one embodiment there is provided a method of treating first-line treatment of metastatic breast cancer in a warm-blooded animal, such as man, which comprises administering to said animal an effective amount of a nucleoside formulation as described herein.

In one embodiment there is provided a method of treating first-line treatment of metastatic breast cancer after failure of adjuvant chemotherapy in a warm-blooded animal, such as man, which comprises administering to said animal an effective amount of a nucleoside formulation as described herein.

In one embodiment there is provided a method of treating first-line inoperable, locally advanced (Stage IIIA or IIIB), or metastatic (Stage IV) non-small cell lung cancer in a warm-blooded animal, such as man, which comprises administering to said animal an effective amount of a nucleoside formulation as described herein.

In one embodiment there is provided a method of treating locally advanced (nonresectable Stage II or Stage III) or metastatic (Stage IV) adenocarcinoma of the pancreas in a warm-blooded animal, such as man, which comprises administering to said animal an effective amount of a nucleoside formulation as described herein.

In one embodiment there is provided a method of treating locally advanced or metastatic bladder cancer in a warm-blooded animal, such as man, which comprises administering to said animal an effective amount of a nucleoside formulation as described herein.

In one embodiment there is provided a method of treating locally advanced or metastatic epithelial ovarian carcinoma in a warm-blooded animal, such as man, which comprises administering to said animal an effective amount of a nucleoside formulation as described herein.

### Use of a Nucleoside Formulation

In one embodiment there is provided the use of a nucleoside formulation as described herein for the manufacture of a medicament for intravenous injection.

In one embodiment there is provided the use of a nucleoside formulation as described herein for the manufacture of a medicament for the treatment of viral infections.

In one embodiment there is provided the use of a nucleoside formulation as described herein for the manufacture of a medicament for the treatment of HIV, ebola, Marburg, coronavirus, hepatitis B, hepatitis C, vaccinia, Epstein-Barr, cytomegalovirus, zikia, herpes simplex and/or varicella zoster virus infection.

In one embodiment there is provided the use of a nucleoside formulation as described herein for the manufacture of a medicament for the treatment of cancer.

In one embodiment there is provided the use of a nucleoside formulation as described herein for the manufacture of a medicament for the treatment of breast, bladder, testicular, pancreatic, ovarian or non-small cell lung cancer.

In one embodiment there is provided the use of a nucleoside formulation as described herein for the manufacture of a medicament for the treatment of breast cancer.

In one embodiment there is provided the use of a nucleoside formulation as described herein for the manufacture of a medicament for the treatment of testicular cancer.

In one embodiment there is provided the use of a nucleoside formulation as described herein for the manufacture of a medicament for the treatment of bladder cancer.

In one embodiment there is provided the use of a nucleoside formulation as described herein for the manufacture of a medicament for the treatment of pancreatic cancer.

In one embodiment there is provided the use of a nucleoside formulation as described herein for the manufacture of a medicament for the treatment of ovarian cancer.

In one embodiment there is provided the use of a nucleoside formulation as described herein for the manufacture of a medicament for the treatment of non-small cell lung cancer.

In one embodiment there is provided the use of a nucleoside formulation as described herein for the manufacture of a medicament for the treatment of metastatic breast cancer.

In one embodiment there is provided the use of a nucleoside formulation as described herein for the manufacture of a medicament for the first-line treatment of metastatic breast cancer.

In one embodiment there is provided the use of a nucleoside formulation as described herein for the manufacture of a medicament for the first-line treatment of metastatic breast cancer after failure of adjuvant chemotherapy.

In one embodiment there is provided the use of a nucleoside formulation as described herein for the manufacture of a medicament for the first-line treatment of patients with inoperable, locally advanced (Stage IIIA or IIIB), or metastatic (Stage IV) non-small cell lung cancer.

In one embodiment there is provided the use of a nucleoside formulation as described herein for the manufacture of a medicament for the treatment of locally advanced (nonresectable Stage II or Stage III) or metastatic (Stage IV) adenocarcinoma of the pancreas.

In one embodiment there is provided the use of a nucleoside formulation as described herein for the manufacture of a medicament for the treatment of locally advanced or metastatic bladder cancer.

In one embodiment there is provided the use of a nucleoside formulation as described herein for the manufacture of a medicament for the treatment of locally advanced or metastatic epithelial ovarian carcinoma.

### Combinations

In one embodiment, the nucleoside formulation as described herein, as a first active ingredient, may be used in combination with a second active ingredient, for example a second anti-cancer medicine. Particularly suitable second active ingredients may be a platinum compound, for example carboplatin or cisplatin.

Herein, where the term “combination” is used it is to be understood that this refers to simultaneous, separate or sequential administration. In one embodiment “combination” refers to simultaneous administration. In one embodiment “combination” refers to separate administration. In one embodiment “combination” refers to sequential administration. Where the administration is sequential or separate, the delay in administering the second component should not be such as to lose the beneficial effect of the combination.

In one embodiment there is provided a nucleoside formulation as described herein in combination with a second active ingredient.

In one embodiment there is provided a nucleoside formulation as described herein in combination with a second active ingredient for use in producing an anti-cancer effect.

In one embodiment there is provided a nucleoside formulation as described herein in combination with a second active ingredient for use in treating breast, bladder, testicular, pancreatic, ovarian or non-small cell lung cancer.

### Kits

In one embodiment there is provided a kit comprising:
a) a nucleoside formulation as described herein;
b) container means for containing said nucleoside formulation.

In one embodiment there is provided a kit comprising:
a) a nucleoside formulation as described herein;
b) container means for containing said nucleoside formulation; and optionally
c) instructions for use.

In one embodiment there is provided a kit comprising a nucleoside formulation as described herein in combination with a second active ingredient.

In one embodiment there is provided a kit comprising:
a) a nucleoside formulation as described herein in a first unit dosage form;
b) a second active ingredient; in a second unit dosage form; and
c) container means for containing said first and second dosage forms.

### DESCRIPTION OF THE FIGURES

In this section RALA refers to the peptide WEARLARALARALARHLARALARALRACEA (SEQ ID_No 1).

FIG. 1 Shows particle size (Z-Average) distribution plots for RALA/dFdCTP nanoparticles (Mole ratio 2.0 (MR2.0)) produced by (i) manual formulation and (ii) automated formulation.

FIG. 2 is a cell cycle analysis of BxPC-3 cells 48 hours following treatment with manually formulated RALA/dFdCTP nanoparticles at various mole ratios. There was a much higher percentage in of cells that were not in G2 phase when treated with RALA/dFdCTP nanoparticles when compared to untreated cells, dFdCTP treated cells and gemcitabine hydrochloride treated cells. This indicates a high level of efficacy of the RALA/dFdCTP nanoparticles. The mole ratio with the lowest percentage of cells in the G2 phase (highest % G0/G1+S) was observed to be MR2.0.

FIG. 3 shows reported data (heat map) following the colony forming assay of Example 6 performed post treatment of both gemcitabine sensitive (BxPC-3) and gemcitabine resistant (PANC-1) cell lines with varying concentrations of RALA/dFdCTP nanoparticles, dFdCTP and gemcitabine hydrochloride. RALA/dFdCTP nanoparticles reduced cell proliferation compared to gemcitabine hydrochloride at low/med/high doses in gemcitabine sensitive cells (BxPC-3) and at med/high does in gemcitabine resistant cells (PANC-1) indicating increased efficacy over gemcitabine hydrochloride or dFdCTP alone, with RALA/dFdCTP nanoparticles exhibiting the best average functionality across both cell lines and associated concentrations when compared with gemcitabine hydrochloride or dFdCTP alone.

FIG. 4 shows RALA/dFdCTP nanoparticle (MR 2.0) functionality in gemcitabine-sensitive (BxPC-3) and gemcitabine-resistant (PANC-1) cells at RALA/dFdCTP EC₅₀ dose. At the EC₅₀ concentration of RALA/dFdCTP nanoparticles, treatment was observed to cause a significantly higher level of yH2AX expression in both BxPC-3 and PANC-1 cells, when compared to dFdCTP or gemcitabine hydrochloride treatments. These results indicate a strong increase in functionality of RALA/dFdCTP nanoparticles over dFdCTP or gemcitabine hydrochloride, as a higher level of DNA damage is possible with a reduced treatment dose.

FIG. 5 shows RALA/dFdCTP nanoparticle (MR2.0) functionality in gemcitabine-sensitive (BxPC-3) cells at RALA/dFdCTP EC₅₀ dose, pre- and post-treatment with 10 μM dipyridamole to induce gemcitabine resistance through blocking nucleotide transport channels to test the ability of RALA/dFdCTP to overcome cellular gemcitabine resistance mechanisms. Dipyridamole treatment was found to reduce the functionality with respect to double strand DNA breaks (yH2AX) of gemcitabine hydrochloride and dFdCTP to a much higher degree than was seen in RALA/dFdCTP nanoparticles. This differentiation is potentially due to the method of entry for RALA/dFdCTP nanoparticles relying on endocytosis rather than entry to the cell via hENT/hCNT channels which dipyridamole blocks. Although the level of functionality of RALA/dFdCTP nanoparticles decreased slightly following treatment with dipyridamole, there remains a significant increase in double strand breaks compared to the other two treatment groups, with levels higher than gemcitabine before pre-dipyridamole treatment.

FIG. 6 shows RALA/dFdCTP nanoparticle (MR2.0) therapeutic efficacy in athymic nude mice (n=6) subcutaneously implanted with 2.5×10⁶ gemcitabine resistant PANC-1 cells. Once tumour size reached a volume of ˜150 mm³, mice were intravenously injected with a single dose of either vehicle (10% w/v trehalose) or 1 mg/kg dFdCTP, gemcitabine or RALA/dFdCTP (MR2.0). Tumours were measured three times weekly until total tumour volume quadrupled (>=600 mm³). (A) Mean tumour volume growth curves for all treatment groups exhibiting the tumour delay following treatment with a single dose of RALA/dFdCTP (MR2.0) when compared to the other treatment groups. (B) Kaplan-Meier survival estimates for the mice treated as part of this study. The increase in survival is clearly observable in the mice that were treated with RALA/dFdCTP (MR2.0). (C) Tumour doubling times as calculated from the tumour volume measurements, indicating the significant increase in doubling time of the RALA/dFdCTP (MR2.0) treated mice.

FIG. 7 shows the increased circulatory half-life of RALA/dFdCTP nanoparticles (MR2.0). C57BL/6 mice (n=6) were intravenously treated with 40 μg of RALA/dFdCTP, dFdCTP or gemcitabine. At predefined timepoints following treatment (0.25/0.5/1/2/4/6/12 h) whole blood was sampled from the tail of the mice and serum extracted via centrifugation. Serum samples were analysed via mass spectroscopy and compared against a standard curve of varying concentrations of the molecule of interest. 7A: PK profile following a single 40 μg dose of RALA/dFdCTP (MR2.0), dFdCTP or gemcitabine injected intravenously into C57BL/6 mice. 7B: Area under the curve (circulatory concentration) of gemcitabine, dFdCTP and RALA/dFdCTP (MR2.0) taken from the respective PK profiles.

FIG. 8 shows the level of in vivo safety biomarkers following intravenous delivery of 40 μg of RALA/dFdCTP nanoparticles (MR2.0), dFdCTP or gemcitabine. Safety markers investigated included; (A) kidney toxicity (creatine), (B,C) liver toxicity (AST & ALT), (D,E,F) immunogenicity markers (TNF-α, IFN-γ and MIP-2).

FIG. 9: shows a measurement of red blood cell (RBC) lysis induced by RALA/dFdCTP as measured in ovine blood. RALA/dFdCTP nanoparticles, or dFdCTP, in an isotonic trehalose solution was added to the RBC suspension, with the equivalent of 10-30 μg dFdCTP added, and the solution incubated for 1 h at 37° C. Following incubation, the erythrocyte suspensions were centrifuged for 90 s at 400 g to form a cell pellet. Absorbance of the supernatant was measured via UV-Vis spectrophotometry (at 541 nm. 1% Triton X-100 was used as a positive control representing 100% haemolysis. Data indicates that there is no detectable haemolytic activity in RBCs when dFdCTP is complexed with RALA but that dFdCTP alone causes >20% haemolysis.

FIG. 10: shows the charge titration curve RALA/dFdCTP nanoparticles. A sample of RALA/dFdCTP nanoparticles that contained 462.5 μg of RALA peptide was titrated with 0.01 M poly(acrylic acid) and the charge of the resultant solution was measured using a multiparameter bench-meter. Non-linear standard curve fit (R²=0.991), equivalence point was calculated and interpolated to reveal volume of titrant added (V=46.33). The volume of titrant (V), concentration of PAA (C) and mass of RALA (W) were used in the equation to calculate the average charge density of the RALA/dFdCTP nanoparticles to be ≤2.0 μmol/mg at 20° C. which shows that the nanoparticles are not cytotoxic due to their charge.

### EXAMPLES

### General Experimental

In the Example section, the following applies:
RALA refers to the peptide WEARLARALARALARHLARALARALRACEA (SEQ ID_No 1) and was obtained from Biomatik, Canada in lyophilised powder form.
dFdCTP refers to gemcitabine triphosphate obtained from Biorbyt®, UK.
dCTP refers to deoxycytidine triphosphate obtained from Biorbyt®, UK.
Gemcitabine refers to gemcitabine hydrochloride obtained from Fluorochem®, UK.
BxPC-3 and PANC-1 cells were obtained from ATCC, USA.
MR stands for mole ratio. The “MR” numbers quoted in the Examples and Figures refers to the number of moles of amphipathic cell penetrating peptide:1 mole of the nucleoside or a phosphate thereof, for example MR 2.0 refers to 2 moles of amphipathic cell penetrating peptide to every 1 mole of nucleoside or a phosphate thereof.

### Example 1: Preparation of RALA Peptide

RALA was reconstituted with molecular grade water to a desired concentration, aliquoted and stored at −20° C. until further use. An aliquot was taken as needed and defrosted on ice. Aliquots were not re-frozen once they had been defrosted.

### Example 2: Nanoparticle Formulation

### 2.1: Manual Formulation

RALA/dFdCTP nanoparticles (final concentration 0.02 mg/mL) were formulated at various RALA:dFdCTP mole ratios by first adding necessary volumes of Ultrapure water to 10 μg of dFdCTP in solution at a concentration of 1 mg/mL, such that the final formulation volume equalled 500 μL. The corresponding volumes of peptide solution at a concentration of 10 mg/mL (Table 1) were added to the diluted dFdCTP solution. The mixture was pipetted up and down approximately 5-10 times to ensure homogenous mixing. Nanoparticles formed spontaneously in solution.

TABLE 1
Manual preparation of Nanoparticles containing different mole ratios

| RALA:dFdCTP | | | | | dFdCTP | Ultrapure | RALA |
|---|---|---|---|---|---|---|---|
| Mole Ratio | dFdCTP | RALA | dFdCTP | RALA | (1 mg/mL) | H₂O | (10 mg/ml) |
| (MR) | [nM] | [nM] | [μg] | [μg] | [μL] | [μL] | [μL] |
| 1.0 | 19.88 | 19.88 | 10.0 | 66.1 | 10.0 | 483.39 | 6.61 |
| 1.2 | 19.88 | 23.85 | 10.0 | 79.4 | 10.0 | 482.06 | 7.94 |
| 1.4 | 19.88 | 27.83 | 10.0 | 92.6 | 10.0 | 480.74 | 9.26 |
| 1.6 | 19.88 | 31.80 | 10.0 | 105.8 | 10.0 | 479.42 | 10.58 |
| 1.8 | 19.88 | 35.78 | 10.0 | 119.1 | 10.0 | 478.09 | 11.91 |
| 2.0 | 19.88 | 39.75 | 10.0 | 132.3 | 10.0 | 476.77 | 13.23 |
| 2.2 | 19.88 | 43.73 | 10.0 | 145.5 | 10.0 | 475.45 | 14.55 |
| 2.4 | 19.88 | 47.70 | 10.0 | 158.7 | 10.0 | 474.13 | 15.87 |
| 2.6 | 19.88 | 51.68 | 10.0 | 172.0 | 10.0 | 472.80 | 17.20 |
| 2.8 | 19.88 | 55.65 | 10.0 | 185.2 | 10.0 | 471.48 | 18.52 |
| 3.0 | 19.88 | 59.63 | 10.0 | 198.4 | 10.0 | 470.16 | 19.84 |

### 2.2: Automated Formulation

RALA/dFdCTP nanoparticles (final concentration 0.02 mg/mL) were formulated at various M RALA:dFdCTP mole ratios by use of an automated microfluidics system (e.g. Precision Nanosystems Ignite NanoAssemblr). Two solutions at the appropriate concentrations (Table 2) were loaded into syringes, and the syringes subsequently loaded into the microfluidics system. Nanoparticles were created using a Total Flow Rate (TFR) of 10 ml/min, and a Flow Rate Ratio (FRR) of 1:1. The resultant solution from the system contained nanoparticles at the concentration of 0.02 mg/mL of dFdCTP.

TABLE 2
Automated preparation of Nanoparticles containing different
mole ratios

| RALA: | | | | | | |
|---|---|---|---|---|---|---|
| dFdCTP | | | | | dFdCTP | RALA |
| Mole Ratio | dFdCTP | RALA | dFdCTP | RALA | conc. | conc. |
| (MR) | [nM] | [nM] | [μg] | [μg] | [mg/mL] | [mg/mL] |
| 1.0 | 19.88 | 19.88 | 10.0 | 66.1 | 0.04 | 0.26 |
| 1.2 | 19.88 | 23.85 | 10.0 | 79.4 | 0.04 | 0.32 |
| 1.4 | 19.88 | 27.83 | 10.0 | 92.6 | 0.04 | 0.37 |
| 1.6 | 19.88 | 31.80 | 10.0 | 105.8 | 0.04 | 0.42 |
| 1.8 | 19.88 | 35.78 | 10.0 | 119.1 | 0.04 | 0.48 |
| 2.0 | 19.88 | 39.75 | 10.0 | 132.3 | 0.04 | 0.53 |
| 2.2 | 19.88 | 43.73 | 10.0 | 145.5 | 0.04 | 0.58 |
| 2.4 | 19.88 | 47.70 | 10.0 | 158.7 | 0.04 | 0.63 |
| 2.6 | 19.88 | 51.68 | 10.0 | 172.0 | 0.04 | 0.69 |
| 2.8 | 19.88 | 55.65 | 10.0 | 185.2 | 0.04 | 0.74 |
| 3.0 | 19.88 | 59.63 | 10.0 | 198.4 | 0.04 | 0.79 |

### Example 3: Lyophilisation of RALA/dFdCTP Nanoparticles

500 μL of RALA/dFdCTP nanoparticles was transferred into a 2 mL lyophilisation vial. Trehalose (20% w/v) was added to the nanoparticle solution in the vial, such that the volume added was equal to half the final reconstitution volume ensuring that once reconstituted in water following lyophilisation, the resultant solution contained 10% w/v trehalose. Following the addition of trehalose, a rubber lyophilisation vial stopper was partially placed on the vial, such that the air-flow notches were still functional to facilitate sublimation during the lyophilisation process. Vials were subsequently loaded into a programmable freeze dryer (e.g. SP Scientific AdVantage Pro) according to the following lyophilisation procedure.

TABLE 3
Lyophilisation Procedure

| 1 | Thermal Treatment | | | | |
|---|---|---|---|---|---|
| | | Shelf Temp. | Ramp | Hold Time | |
| | Step | [° C.] | Time [min] | [min] | |
| | 1 | 5 | 60 | 10 | |
| | 2 | −35 | 90 | 0 | |
| 2 | Drying Steps | | | | |
| | | Shelf Temp. | Ramp | Hold Time | Vacuum |
| | Step | [° C.] | Time [min] | [min] | [mTorr] |
| | 3 | −35 | 0 | 180 | 120 |
| | 4 | −30 | 60 | 0 | 190 |
| | 5 | −30 | 0 | 180 | 190 |
| | 6 | −25 | 60 | 0 | 190 |
| | 7 | −25 | 0 | 180 | 190 |
| | 8 | 20 | 120 | 0 | 190 |
| | 9 | 20 | 0 | 360 | 190 |

| 3 | Storage | | |
|---|---|---|---|
| | Shelf Temp | 20 | ° C. |
| | Vacuum | 50 | mTorr |

| | | |
|---|---|---|
| 4 | Backfilling | None |
| 5 | Stoppering | Vials Stoppered under Vacuum |

### Example 4: Nanoarticle Size and Charge Analysis

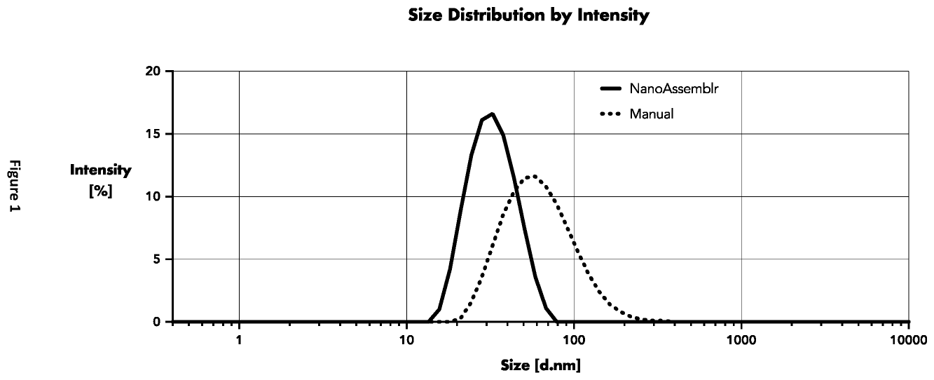

Z-Average particle size measurements and polydispersity (Pdl) of RALA/dFdCTP nanoparticles were performed using Dynamic Light Scattering (DLS) in order to obtain particle size and charge distributions. Surface charge measurements of the RALA nanoparticles were determined by Laser Doppler Velocimetry. The zeta potential of the particles was measured using disposable foldable zeta cuvettes. Zeta cuvettes for the measurement of zeta potential were first washed with 70% ethanol, followed by two rinses with double distilled H₂O prior to loading the sample. 50 μL of neat sample was used for size measurements, subsequently diluted to 1 mL with UltraPure water and then 700-800 μL of diluted sample was used for determination of zeta potential. The nanoparticles were made up at a range of mole ratios (MR 1.4, MR 2.0-3.0) using at least 1 μg of dFdCTP in each sample. Nanoparticles were analysed on a Zetasizer-Nano-ZS (Malvern Instruments) with DTS software (Malvern Instruments, UK) and the results are shown in FIG. 1 and Table 4.

TABLE 4
RALA/dFdCTP Particle Size (Z-Average), Particle Charge
(Zeta Potential) and Polydispersity Index (PdI)

| | Nanoparticle | | Zeta | |
|---|---|---|---|---|
| RALA/dFdCTP | formulation | Z-Average | Potential | Polydispersity |
| Particles | method | [nm] | [mV] | Index |
| Mole Ratio 1.4 | Manual | 2943 ± 538 | −6.8 ± 1.1 | 0.619 ± 0.147 |
| Mole Ratio 2.0 | Manual | 74 ± 2 | 35.7 ± 1.2 | 0.436 ± 0.049 |
| Mole Ratio 2.0 | AUTO | 27 ± 3 | 32.4 ± 2.4 | 0.221 ± 0.056 |
| Mole ratio 2.2 | Manual | 71 ± 1 | 38.3 ± 2.0 | 0.350 ± 0.013 |
| Mole ratio 2.4 | Manual | 67 ± 5 | 41.4 ± 1.2 | 0.413 ± 0.040 |
| Mole ratio 2.6 | Manual | 61 ± 3 | 43.2 ± 1.2 | 0.352 ± 0.009 |
| Mole ratio 2.8 | Manual | 57 ± 4 | 42.8 ± 2.3 | 0.343 ± 0.011 |
| Mole ratio 3.0 | Manual | 67 ± 4 | 42.6 ± 1.1 | 0.319 ± 0.037 |

Results indicate that a mole ratio of ≥2.0 facilitates the formation of nanoparticles that meet critical quality attributes (Z-Average<150 nm, Zeta Potential>+10 mV, Pdl<0.500). Similarly, with the use of an automated microfluidic mixer, these particle physiochemical characteristics can be further enhanced (Z-Average<100 nm, Zeta Potential>+10 mV, Pdl<0.300).

### Example 5: Cell Cycle Analysis

1.5×10⁵ BxPC-3 cells were plated in 6-well plates (Nunc, UK) and left to adhere for 24 h. Cells were transferred to serum free media for 24-h, to ensure synchronisation of cells. Subsequently, cells were incubated with RALA/dFdCTP nanoparticles at a concentration of 2 μM (1 μg) for 5 h before removal of treatment media and the addition of fresh complete media. 48 h following treatment, cells were trypsinsed and fixed in ice-cold 70% ethanol for 1 hour at 4° C. Cells were washed twice in Phosphate Buffered Saline (PBS) for 5 min, before resuspension in 500 μL PBS and the addition of 50 μL of 100 μg/ml RNase (Invitrogen, UK). 10 minutes prior to running samples on fluorescence-activated cell sorting (FACS), 10 μl of a 100 μg/ml stock of propidium iodide (PI) was added. Controls include: untreated; dCTP (negative control); RALA/dCTP (RALA control); dFdCTP (drug control); and gemcitabine (comparator control).

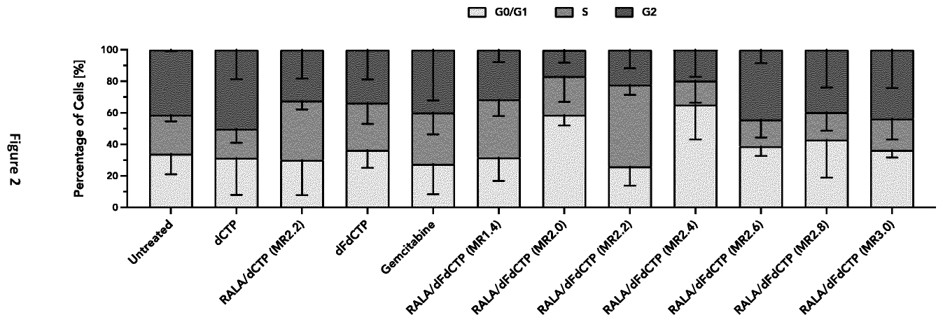

A cell cycle analysis following treatment with RALA/dFdCTP was undertaken. If dFdCTP had been successfully delivered to cells, it would be incorporated during DNA polymerase, preventing DNA chain elongation. This would ultimately prevent cells from actively proliferating. This means that during cell cycling, the cycle will be arrested at the S-phase. Therefore, if there are a higher percentage of the cells in the (G0/G1+−S) phases than compared to the G2 phase, it is an indicator that dFdCTP has affected the cell cycle. The results are shown in FIG. 2 and Table 5.

TABLE 5
Cell cycle analysis on BxPC-3 cells 48 hours following treatment
with RALA/dFdCTP at various mole ratios.

| Treatment (manual formulations) | Cells in (G0/G1 + S) Phase [%] |
|---|---|
| Untreated | 58.7 |
| dCTP | 49.9 |
| RALA/dCTP (MR2.2) | 67.7 |
| dFdCTP | 66.4 |
| Gemcitabine | 60.2 |
| RALA/dFdCTP (MR1.4) | 68.5 |
| RALA/dFdCTP (MR2.0) | 83.2 |
| RALA/dFdCTP (MR2.2) | 77.8 |
| RALA/dFdCTP (MR2.4) | 80.4 |
| RALA/dFdCTP (MR2.6) | 55.8 |
| RALA/dFdCTP (MR2.8) | 60.4 |
| RALA/dFdCTP (MR3.0) | 56.3 |

### Example 6: Clonogenic Assay

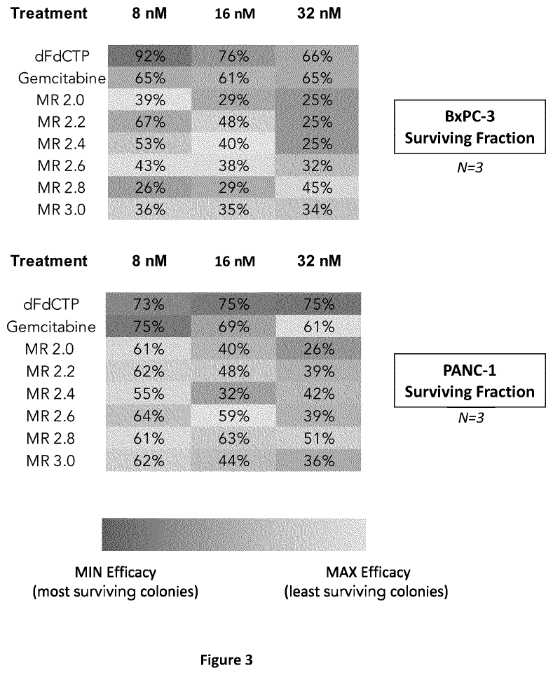

1×10⁵ (BxPC-3 and PANC-1) cells were treated with 8/16/32 nM of gemcitabine, dFdCTP or RALA/dFdCTP manually formulated nanoparticles of a variety of mole ratios. 5 hours following transfection, cells were trypsinised, counted and reseeded at low densities (BxPC-3—300 & 600 cells/well and PANC-1—400 & 800 cells/well). Colonies were allowed to form over 14 days before staining with crystal violet and subsequent counting. The results are shown in FIG. 3.

### Example 7: RALA/dFdCTP Functionality (γH2AX)

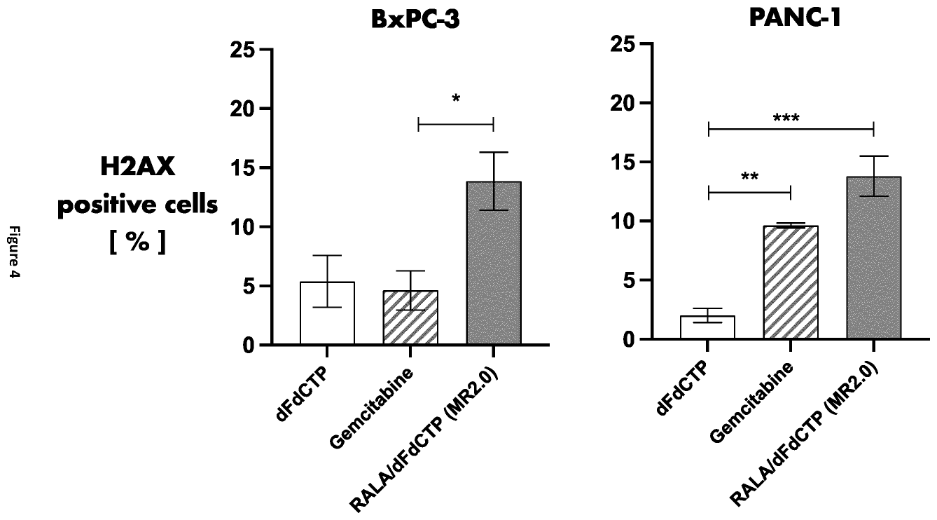

In order to test the functionality of RALA/dFdCTP nanoparticles, transfection of BxPC-3 (gemcitabine-susceptible) or PANC-1 (gemcitabine resistant) cells was performed. Initially, 1.5×10⁵ cells were plated in 6-well microplates (Nunc, UK) and left to adhere for 24 h. Cells were subsequently incubated with manually formulated RALA/dFdCTP (MR 2.0), dFdCTP or gemcitabine hydrochloride at an EC₅₀ concentration (previously determined via clonogenic assay) for 5 h, before removal of transfection media, and the addition of complete cell culture media. 2 h and 24 h post-transfection, cells were trypisined, fixed in 4% formaldehyde (Sigma, UK) before permeabilization overnight at −20° C. in methanol. Cells were blocked in PBS containing 10% fetal bovine serum (FBS). Samples were incubated for 2 h in primary γH2AX (phosphS139) antibody (1:100 dilution in PBS containing 10% FBS) (Abcam, UK). Subsequently, cells were washed in PBS for 5 min, before incubation with a fluorescein isothiocyanate (FITC) tagged secondary γH2AX antibody (Abcam, UK) at room temperature for 1 h (1:50 dilution in PBS containing 10% FBS). Washes were repeated. The percentage of γH2AX expressing cells were measured using FACS Calibur (BD Biosciences, UK) and analysed using FlowJo analysis software. The results are shown in FIG. 4.

### Example 8: Overcoming Gemcitabine Resistance Using RALA/dFdCTP

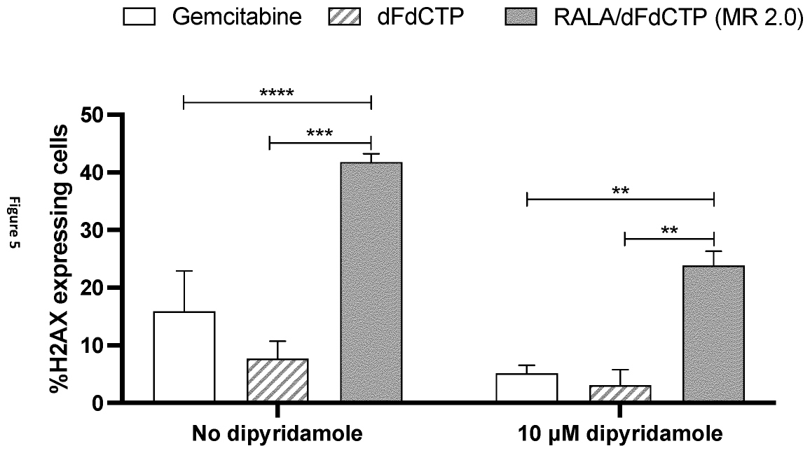

1.5×10⁵ of the gemcitabine-susceptible cell line BxPC-3 were plated in 6-well microplates (Nunc, UK) and left to adhere for 24 h. A subset of cells were subsequently treated with 10 μM of dipyridamole (Sigma, UK), a nucleotide transport channel (hENT/hCNT) inhibitor, before transfection with manually formulated RALA/dFdCTP (MR 2.0), dFdCTP or gemcitabine hydrochloride, at an EC₅₀ concentration (previously determined via clonogenic assay) for 5 h. Determination of subsequent functionality was carried out by investigating levels of γH2AX post-transfection via the procedure outlined in Example 7. The results are shown in FIG. 5.

### Example 9: In Vivo Efficacy

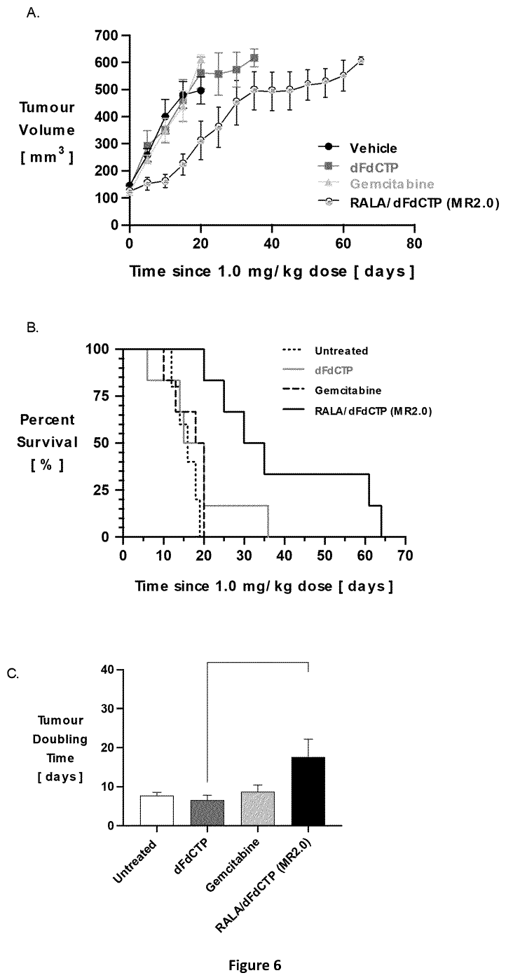

24 female athymic (nude) mice were weighed and under anaesthesia (isoflurane) subcutaneously implanted with 2×106 PANC-1 cells in volume of 100 μl PBS on the right flank. For treatments to commence tumours were required to establish and reach a volume of 100-150 mm³. Once the tumours reached the required volume mice were then treated with a single 1 mg/kg intravenous dose of either 10% Trehalose (vehicle control), RALA/dFdCTP (MR2.0), dFdCTP or gemcitabine in 100 μl total volume (n=6 per treatment arm). Subsequently, tumour volumes and mice weights were monitored and recorded 3 times weekly. Once tumours reached endpoint (specified as volume of 600 mm3), mice were sacrificed. The results of this study are presented in FIG. 6.

### Example 10: Pharmacokinetic and Safety Profile

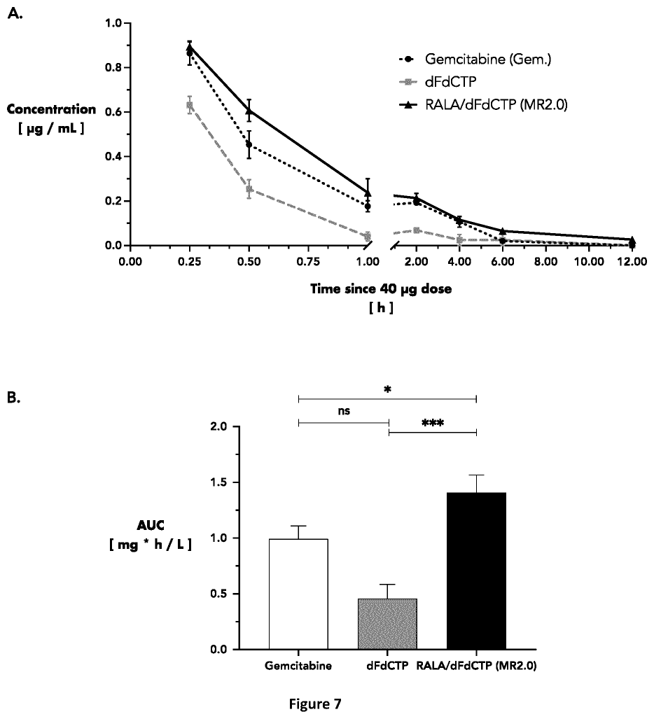

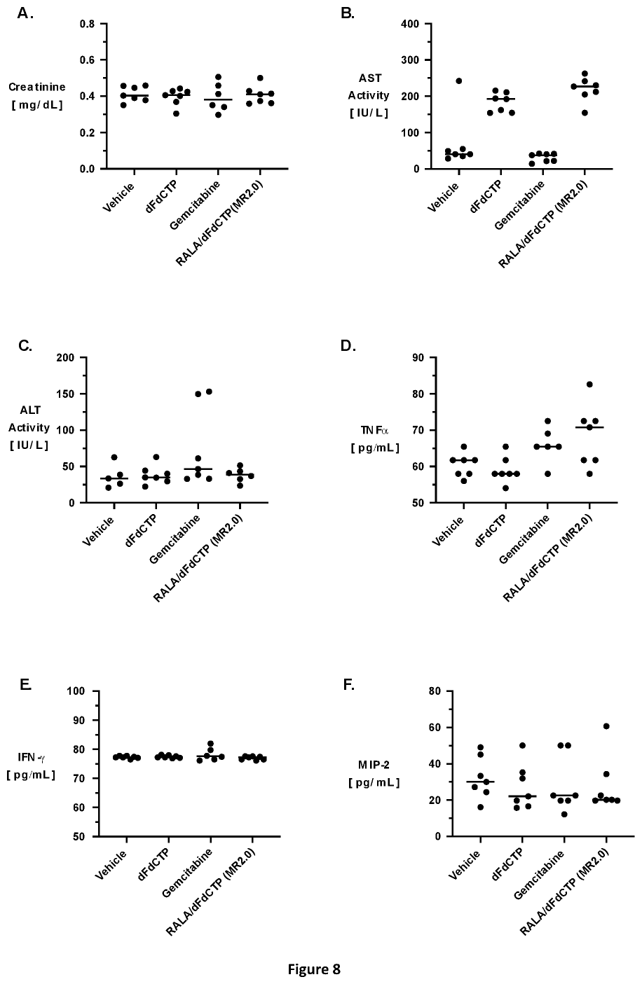

C57BL/6 mice (n=7 per treatment arm) received a single i.v injection (40 μg) of gemcitabine, dFdCTP or RALA/dFdCTP (MR2.0). Mice were bled at various time points and plasma extracted for analysis. dFdCTP or gemcitabine content in the collected plasma was measured via Mass Spectrometry. Terminal cardiac bleeds were performed at endpoint and serum extracted for safety profile characterisation via multiplex and standard ELISAs for kidney toxicity (creatinine), liver toxicity (AST, ALT) and immunogenicity markers (TNF-α, IFN-γ and MIP-2). The results are presented in FIG. 7 and FIG. 8.

### Example 11: Haemolysis Assay

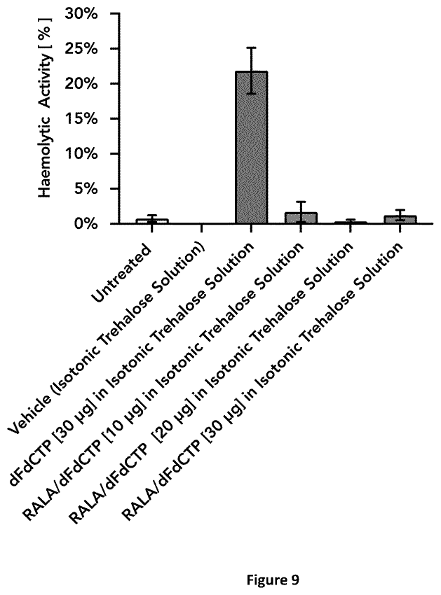

Defibrinated ovine whole blood (5 ml, TCS Biosciences Ltd, UK) was centrifuged at 500 g in citrate-phosphate buffer (15 ml, 0.1 M citric acid, C₆HsO₇, and 0.2 M disodium hydrogen phosphate, Na₂HPO₄) for 20 min to separate erythrocytes. The erythrocytes were subsequently washed three times with 20 mL of citrate-phosphate buffer by centrifugation and re-suspended in citrate-phosphate buffer solution at a concentration of 1×108 cells/mL. A Countess II automated cell counter (ThermoFisher, UK) was used to count erythrocytes by first diluting the erythrocyte suspension 1:10,000 in buffer. Subsequently, isotonic solutions (of osmolality in the range 290-310 mOsmol/kg) of RALA/dFdCTP (MR2.0) nanoparticles mixed with trehalose, or dFdCTP and trehalose was added to the erythrocyte suspension, with the equivalent of 10-30 μg dFdCTP added, and the solution incubated for 1 h at 37° C. Following incubation, the erythrocyte suspensions were centrifuged for 90 s at 400 g to form a cell pellet. Absorbance of the supernatant was measured via a Nanodrop 2000c UV-Vis spectrophotometer (Thermo Scientific, UK) at 541 nm. 1% Triton X-100 was used as a positive control representing 100% haemolysis; and citrate-phosphate buffer at pH 7.4 was used as a negative control. Percentage haemolytic activity was calculated using the Equation 1 and the results shown in FIG. 9.

$$\frac{( {A}_{541} of sample - {A}_{541} of negative control )}{( {A}_{541} of positive control - {A}_{541} of negative control )} \times 100 \% \qquad Eq . l$$

### Example 12: Charge Density

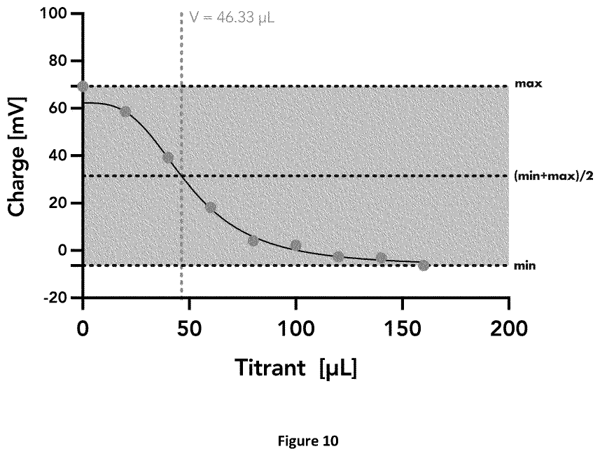

The charge density of RALA/dFdCTP (MR2.0) nanoparticles was measured by polyelectrolytic titration using the methods described in Ritz et al, Biomacromolecules. 2015 Apr. 13; 16(4):1311-21. doi: 10.1021/acs.biomac.5b00108. Epub 2015 Apr. 3 and Weiss et al, J Nanobiotechnology. 2021 Jan. 6; 19(1):5. doi: 10.1186/s12951-020-00747-7. The polyelectrolytic titration was performed using poly(acrylic acid) (PAA) 0.01 M at pH 7.4. PAA was added to RALA/dFdCTP (MR2.0) nanoparticles whilst measuring the charge to create a sigmoidal curve of which the volume (V) can be derived from the equivalence point. The mass of peptide in the system (w) and concentration of PAA (0.01 M) used, was used to calculate the mean charge density of the nanoparticle (Qₑₖ). The results are shown in FIG. 10.
Qek=V×c/w
V=volume of titrant=46.33 μl
c=concentration of titrant—0.01 M
w=mass of RALA=0.4625 mg
Qek=46.33×(0.01/0.4625)
Qek=1.002

### STATEMENTS

Statement 1. A nucleoside formulation comprising:
(i) a nucleoside or a phosphate thereof; and
(ii) an amphipathic cell penetrating peptide comprising or consisting of the amino acid sequence:

(SEQ ID_No 1)
WEARLARALARALARHLARALARALRACEA

or a sequence with at least 80% sequence identity or homology.
   Statement 2. A nucleoside formulation as stated in statement 1 wherein the nucleoside or a phosphate thereof is selected from didanosine, vidarabine, galidesivir, remdesivir, cytarabine, gemcitabine, emtricitabine, lamivudine, zalcitabine, uyabacavir, aciclovir, entecavir, stavudine, telbivudine, zidovudine, idoxuridine or trifluridine or a phosphate thereof.
   Statement 3. A nucleoside formulation as stated in statement 1 or statement 2 wherein the nucleoside or a phosphate thereof is gemcitabine or a phosphate thereof.
   Statement 4. A nucleoside formulation as stated in any one of the preceding statements wherein the nucleoside or a phosphate thereof is gemcitabine triphosphate.
   Statement 5. A nucleoside formulation as stated in any one of the preceding statements wherein the phosphate comprises a P(O)(OH)₂O— group.
   Statement 6. A nucleoside formulation as stated in any one of the preceding statements wherein the amphipathic cell penetrating peptide comprises the amino acid sequence

(SEQ ID_No 1)
WEARLARALARALARHLARALARALRACEA.

Statement 7. A nucleoside formulation as stated in any one of the preceding statements wherein the amphipathic cell penetrating peptide consists of the amino acid sequence

(SEQ ID_No 1)
WEARLARALARALARHLARALARALRACEA.

Statement 8. A nucleoside formulation as stated in any one of the preceding statements wherein the mole ratio of nucleoside or a phosphate thereof:amphipathic cell penetrating peptide is 1:1-3.
Statement 9. A nucleoside formulation as stated in any one of the preceding statements wherein the mole ratio of nucleoside or a phosphate thereof:amphipathic cell penetrating peptide is about 1:2.
Statement 10. A nucleoside formulation as stated in any one of the preceding statements additionally comprising a bulking agent.
Statement 11. A nucleoside formulation as stated in statement 10 wherein the bulking agent comprises trehalose, sucrose, mannose and/or dextrose.
Statement 12. A nucleoside formulation as stated in any one of the preceding statements wherein the formulation is a nanoparticle formulation.
Statement 13. A method of preparing a nanoparticle formulation as stated in statement 12 which comprises (i) formulating a solution of WEARLARALARALARHLARALARALRACEA (SEQ ID_No 1) or a sequence with at least 80% sequence identity or homology with (ii) a nucleoside or a phosphate thereof, in an automated controlled mixing system, particularly an automated microfluidics system.
Statement 14. A nucleoside formulation as stated in any one of statements 1-12 for use as a medicament.
Statement 15. A nucleoside formulation as stated in any one of statements 1-12 for use in the treatment of viral infections.
Statement 16. A pharmaceutical composition which comprises a nucleoside formulation as stated in any one of statements 1-12 for use in the treatment of cancer.
Statement 17. A method of treating breast, bladder, testicular, pancreatic, ovarian or non-small cell lung cancer in a warm-blooded animal, such as man, which comprises administering to said animal an effective amount of a nucleoside formulation as stated in any one of statements 1-12.
Statement 18. The use of a nucleoside formulation as stated in any one of statements 1-12 for the manufacture of a medicament for the treatment of HIV, ebola, Marburg, coronavirus, hepatitis B, hepatitis C, vaccinia, Epstein-Barr, cytomegalovirus, zikia, herpes simplex and/or varicella zoster virus infection

1. A nucleoside formulation comprising:
(i) gemcitabine or a phosphate thereof; and
(ii) an amphipathic cell penetrating peptide comprising or consisting of the amino acid sequence:
(SEQ ID_No 1)
WEARLARALARALARHLARALARALRACEA
or a sequence with at least 80% sequence identity or homology.

2. The nucleoside formulation as claimed in claim 1 wherein the phosphate comprises a P(O)(OH)₂O— group.

3. The nucleoside formulation as claimed in claim 1 wherein gemcitabine or a phosphate thereof is gemcitabine triphosphate.

4. The nucleoside formulation as claimed in claim 1 wherein the amphipathic cell penetrating peptide comprises the amino acid sequence WEARLARALARALARHLARALARALRACEA (SEQ ID_No 1).

5. The nucleoside formulation as claimed in claim 1 wherein the amphipathic cell penetrating peptide consists of the amino acid sequence WEARLARALARALARHLARALARALRACEA (SEQ ID_No 1).

6. The nucleoside formulation as claimed in claim 1 wherein the mole ratio of gemcitabine or a phosphate thereof:amphipathic cell penetrating peptide is 1:1-3.

7. The nucleoside formulation as claimed in claim 1 wherein the mole ratio of gemcitabine or a phosphate thereof:amphipathic cell penetrating peptide is about 1:2.

8. The nucleoside formulation as claimed in claim 1 additionally comprising trehalose, sucrose, mannose and/or dextrose.

9. The nucleoside formulation as claimed in claim 1 additionally comprising trehalose.

10. The nucleoside formulation as claimed in claim 1 comprising:
(i) 0.1-1% w/w gemcitabine or a phosphate thereof;
(ii) 1-10% w/w an amphipathic cell penetrating peptide comprising or consisting of the amino acid sequence WEARLARALARALARHLARALARALRACEA (SEQ ID_No 1) or a sequence with at least 80% sequence identity or homology; and
(iii) 89-99% w/w trehalose, sucrose, mannose and/or dextrose.

11. The nucleoside formulation as claimed in claim 10 formulated as an isotonic formulation in water.

12. A nucleoside formulation comprising:
(i) 0.2-0.5% w/w gemcitabine triphosphate;
(ii) 3.5-4.5% w/w an amphipathic cell penetrating peptide consisting of the amino acid sequence WEARLARALARALARHLARALARALRACEA (SEQ ID_No 1);
(iii) 95-97% w/w trehalose;
formulated as an isotonic formulation in water with an osmolality of 290-310 mOsmol/kg.

13. The nucleoside formulation as claimed in claim 1 wherein the formulation is a nanoparticle formulation.

14. The nucleoside formulation as claimed in claim 13 wherein the mean surface charge density of the nanoparticles is ≤2 μmol/mg at 20° C.

15. A method of preparing a nucleoside formulation as claimed in claim 1 which comprises (i) formulating a solution of WEARLARALARALARHLARALARALRACEA (SEQ ID_No 1) or a sequence with at least 80% sequence identity or homology with (ii) gemcitabine or a phosphate thereof, in an automated controlled mixing system, particularly an automated microfluidics system.

16. The nucleoside formulation as claimed in claim 1 for use as a medicament.

17. The nucleoside formulation as claimed in claim 1 for use in an intravenous injection.

18. A pharmaceutical composition which comprises a nucleoside formulation as claimed in claim 1 for use in the treatment of cancer.

19. A method of treating breast, bladder, testicular, pancreatic, ovarian or non-small cell lung cancer in a warm-blooded animal, such as man, which comprises administering to said animal an effective amount of a nucleoside formulation as claimed in claim 1.


SEQUENCE LISTING (1 sequence)

SEQ ID NO: 1 - protein, 30 aa, synthetic construct
WEARLARALARALARHLARALARALRACEA